(19) United States
(12) Patent Application Publication
(10) Pub. No.: US 2005/0171198 A1
(43) Pub. Date: Aug. 4, 2005

(54) SODm therapy for treatment, prevention, inhibition and reversal of inflammatory disease

(75) Inventor: Daniela Salvemini, Chesterfield, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignees: Metaphore Pharmaceuticals, Inc.; Pharmacia Corporation

(21) Appl. No.: 10/971,799

(22) Filed: Oct. 21, 2004

Related U.S. Application Data
(63) Continuation-in-part of application No. 09/997,974, filed on Nov. 30, 2001, now abandoned.
Application No. 09/997,974 is a continuation-in-part of application No. 09/634,152, filed on Aug. 9, 2000, now Pat. No. 6,395,725.
Application No. 09/634,152 is a division of application No. 09/057,831, filed on Apr. 9, 1998, now Pat. No. 6,180,620.
(60) Provisional application No. 60/050,402, filed on Jun. 20, 1997.

(51) Int. Cl. A61K 31/35; A01N 43/16

(52) U.S. Cl. 514/492

(57) ABSTRACT

The present invention relates to the use of a manganese complex of a heterocyclic pentaazacyclopentadecane ligand, which is effective as a catalyst for dismutating superoxide, particularly in treating, preventing, inhibiting and reversing inflammatory disease.

### CROSS-REFERENCE TO RELATED APPLICATIONS

This application is a continuation-in-part application of co-pending U.S. application Ser. No. 09/997,974 filed Nov. 30, 2001, which is a continuation-in-part application of U.S. application Ser. No. 09/634,152 filed Aug. 9, 2000, now U.S. Pat. No. 6,395,725 issued May 28, 2002, which is a divisional application of U.S. application Ser. No. 09/057,831 filed Apr. 9, 1998, now U.S. Pat. No. 6,180,620 issued Jan. 30, 2001, which is a non-provisional application of U.S. Provisional Application Ser. No. 60/050,402 filed Jun. 20, 1997. Each of the above references are incorporated herein by reference in their entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF THE INVENTION

1. Field of the Invention

The present invention relates to the use of a manganese complex of a heterocyclic pentaazacyclopentadecane ligand, which is effective as a catalyst for dismutating superoxide, particularly in treating, preventing, inhibiting and reversing inflammatory disease.

2. Description of Related Art

Inflammatory disease is any disease marked by inflammation, which is a localized protective response elicited by injury or destruction of tissues and serves to destroy, dilute, or wall off both the injurious agent and the injured tissue. Inflammation is characterized in the acute form by the classical signs of pain, heat, redness, swelling and loss of function. Inflammation occurs when, upon injury, recruited polymorphonuclear leukocytes release reactive oxygen species (“ROS”) in oxidative bursts resulting in a complex cascade of events. Histologically, it involves a complex series of events, including dilation of arterioles, capillaries, and venules, with increased permeability and blood flow; exudation of fluids, including plasma proteins; and leukocytic migration into the inflammatory focus. Inflammatory diseases include, arthritis, inflammatory bowel disease, asthma, psoriasis, lupus and other autoimmune diseases. The inflammation associated with inflammatory diseases may be caused by a multitude of inciting events, including radiant, mechanical, chemical, infectious, and immunological stimuli.

One of the most prominent inflammatory diseases is arthritis. Arthritis is a term that refers to a group of more than 100 diseases that cause joint swelling, tissue damage, stiffness, pain (both acute and chronic), and fever. Arthritis can also affect other parts of the body other than joints including but not limited to: synovium, joint space, collagen, bone, tendon, muscle and cartilage, as well as some internal organs. The two most common forms of arthritis are osteoarthritis (“OA”) and rheumatoid Arthritis (“RA”). RA is the most severe of these two forms in terms of pain; while OA is by far the most common form. RA is a systematic, inflammatory, autoimmune disease that commonly affects the joints, particularly those of the hands and feet. The onset of rheumatoid arthritis can occur slowly, ranging from a few weeks to a few months, or the condition can surface rapidly in an acute manner.

At the cellular level, inflammatory diseases are characterized by an accumulation of cytokines such as TNF-α, IL-1β, IL-6, IL-9, IL-11, IL-15, IL-5 and several belonging to the interferon family, as well as inflammatory cells (e.g., eosinophils, neutrophils, and macrophages). For arthritis specifically, these chemicals build up in the synovial fluid during an arthritic flare-up. Many of these cytokines and mediators released from inflammatory cells cause cell and tissue damage. Additionally, another significant characteristic of the inflammatory response associated with arthritis and other diseases like lupus is a process called autoimmunity. Autoimmunity occurs when, for example, T-cells mistake the body's own collagen cells as foreign antigens and set off a series of events to clear the erroneously perceived threat. This results in an attack of the body's own cells by its immune system. Autoimmunity is particularly associated with rheumatoid arthritis and lupus. The immune response associated with arthritic flare-up is also characterized by oxidative and nitrosative stress and poly ADP-ribose synthetase (“PARS”) activity. A number of strategies have been developed to suppress autoimmune diseases, most notably drugs which nonspecifically suppress the immune response.

Aspirin and related nonsteroidal anti-inflammatory drugs (“NSAIDs”) are widely used to treat inflammatory diseases, but this class of compounds has inherent problems and limitations. The use of NSAIDs commonly causes stomach upset, headache, drowsiness, easy bruising, high blood pressure, and fluid retention. NSAIDs that are nonselective for the cyclooxygenase 2 (“COX-2”) enzyme produced in inflammation also inhibit constitutive cyclooxygenase 1 (“COX-1”) enzyme, causing undesirable damage to the gastric mucosa and leading to dyspepsia, gastritis, or even gastric ulcers. Gastric ulcers may cause bleeding that goes undetected and results in anemia. Furthermore, NSAIDs may affect the function of platelets, impairing the ability of blood to clot.

NSAIDs operate by inhibiting cyclooxygenase enzymes and thereby the synthesis of prostaglandins. Prostaglandins sensitize pain receptors, lowering the pain threshold and making normal stimuli, such as touch and stretch sensations, painful. NSAIDs can be quite effective at returning the lowered pain threshold associated with inflammatory diseases to normal but do not elevate the pain threshold.

A second class of pain relievers, opioids, are also used to treat the pain associated with inflammatory disease. Opioids operate by mimicking natural peptides such as enkephalins and endorphins to stimulate one or more of the μ-, δ- and κ-receptor systems in the nervous system. Opioids elevate the pain threshold so that normally painful stimuli are perceived as less painful or even euphoric. Opioids are commonly used in the clinical management of severe pain, including chronic severe pain of the kind experienced by cancer patients.

Capsaicin and its derivatives operate by depleting local stores of substance P, a neuropeptide involved in the transmission of pain impulses and are used in several OTC analgesic products.

Each of these classes of compounds has inherent problems and limitations. The opioid analgesics are antagonized by analogous N-allyl compounds such as naloxone; the NSAID analgesics are not. As described above, NSAIDs that are nonselective for the cyclooxygenase 2 produced in inflammation (COX-2) also inhibit constitutive cyclooxygenase 1 (COX-1), causing undesirable damage to the gastric mucosa. They have limited effectiveness as analgesics in lowering an elevated threshold to normal and are generally used for mild to moderate pain. They are also ineffective drugs for elevation of the pain threshold above normal levels, which prevents their use in pain such as surgical pain where an underlying pathological condition has not elevated the pain threshold.

Opioids have problems with tolerance and dependency, so that over a course of therapy increasing dosages of compound are required to achieve the same level of analgesia, and cessation of opioid administration when analgesia is no longer needed elicits a withdrawal syndrome with unpleasant and potentially serious symptoms. The dependency and withdrawal syndrome both make it difficult for the clinician to discontinue opioid therapy even when the opioids are no longer effective in relieving pain because of the development of tolerance. Narcotic induced hyperalgesia (NIH) can also develop in association with tolerance to the opioids. All of these factors limit the usefulness of opioids in the management of chronic severe pain, despite their potency.

No adequate strategy has been devised to overcome the development of opioid tolerance and provide an ongoing approach to the management of chronic severe pain. Mechanisms of tolerance are not well understood but are known to involve the NMDA receptor, since the NMDA receptor antagonist MK-801 has been shown in rats to prevent morphine tolerance. NMDA stimulates nitric oxide synthase (NOS) and NOS has been observed histochemically in tissues that contain opioid receptors and are important in the pain response, such as the amygdala, cortical gray matter, and the substantia gelatinosa of the spinal cord. Non-selective NOS inhibitors such as NG-nitroarginine prevent and reverse morphine tolerance. However, nonselective inhibition of NOS is associated with a vast array of undesirable side effects, including hypertension, increased platelet and white blood cell reactivity, decreased cerebral blood flow, and gastrointestinal and renal toxicity.

Capsaicin and some of its derivatives, in addition to producing analgesia, also elicit a burning sensation. This effect is responsible for the pungency of hot peppers (Capscum spp.) and limits the applicability of many members of this series of compounds.

In moderate to advanced cases of arthritis and other inflammatory diseases, corticosteroids, gold salts, anti-malarials and systemic immunosuppressants are used in addition to or in lieu of NSAIDs and opioids. Corticosteroids are a very effective drug for the treatment of arthritis as well as other inflammatory diseases and are the most potent anti-inflammatory agents known. Therefore, corticosteroids are the most widely used anti-inflammatory drugs for both acute and chronic inflammation. For example, glucocorticoids are the most widely used immunosuppressive drugs and are pharmacologically the most potent antiinflammatory agents known. Corticosteroids are used orally, parenterally, and frequently, intra- and peri-articularly, i.e., injections in and around joints and joint cavities. However, the side effects associated with corticosteroid use can be severe. Unfortunately the glucocorticoid side effects profile occurs at doses much lower than those required for an anti-inflammatory effect. And, because both beneficial and detrimental effects are mediated by the same glucocorticoid receptor, it is difficult to separate anti-inflammatory efficacy from fluid and electrolyte abnormalities, hypertension, hyperglycemia, increased susceptibility to infection, osteonecrosis, osteoporosis, myopathy, behavioral disturbances, cataracts, growth arrest, fat redistribution, striae, ecchymoses, acne, and hirsutism.

RA is a common human autoimmune disease characterized by chronic inflammation of the synovial joints and by subsequent progressive destruction of articular tissue. Although the initiating event in RA has not yet been defined, a growing body of evidence indicates that superoxide anions (O₂^{•-}, the one-electron reduction product of oxygen) perpetuate the chronic inflammatory state associated with RA.

In addition to O₂^{•-}, reactive oxygen species (“ROS”) also include the hydroxyl radical, OH^{•}, and nitric oxide, NO^{•}, as well as other species. Besides RA, reactive oxygen metabolites derived from the superoxide anion are postulated to contribute to tissue pathology in a number of inflammatory diseases, such as reperfusion injury (particularly for the intestine, liver, heart and brain), inflammatory bowel disease, osteoarthritis, atherosclerosis, hypertension, cancer, skin disorders (e.g. psoriasis, dermatitis), organ transplant rejections, chemotherapy and radiation-induced side effects, pulmonary disorders (e.g., chronic obstructive pulmonary disease (“COPD”), asthma, influenza, stroke, burns, AIDs, malaria, parkinson's disease and trauma. See, e.g., Simic, M. G., et al, “Oxygen Radicals in Biology and Medicine”, Basic Life Sciences, Vol. 49, Plenum Press, New York and London, 1988; Weiss J. Cell. Biochem., 1991 Suppl. 15C, 216 Abstract C110 (1991); Petkau, A., Cancer Treat. Rev. 13, 17 (1986); McCord, J. Free Radicals Biol. Med., 2, 307 (1986); and Bannister, J. V. et al, Crit. Rev. Biochem., 22, 111 (1987).

ROS are produced in vivo through normal cellular respiration and natural biological signaling and defense mechanisms. Although cellular respiration is important to maintaining life, these highly reactive byproduct molecules have been implicated in a wide range of diseases and conditions. For example, during inflammation, recruited polymorphonuclear leukocytes release ROS during the oxidative burst of phagocytosis. However, during chronic and/or systemic inflammation, the body's ability to control the levels of ROS, specifically the superoxide anion radical, becomes overwhelmed. Llesuy et al., Free Radical Biology and Medicine, 16(4), 445-451 (1994); Taylor et al., Journal of Critical Care, 10(3), 122-136 (1995). The rampant oxidative stress that occurs during this stage of sepsis quickly reduces the levels and/or activities of the body's natural antioxidants (e.g., ascorbate, superoxide dismutase, catalase, glutathione peroxidase, vitamin E) and lipid peroxides begin to accumulate. Additionally, endogenous catecholamines and cortisol may be inactivated leading to a drop in blood pressure and an increase in vascular permeability. See Macarthur et al., Inactivation of Catecholamines by Superoxide Gives New Insights on the Pathogenesis of Septic Shock, PNAS, Vol. 97, No. 17, 9753-9758 (Aug. 15, 2000).

Sources of ROS in inflammatory joints are numerous. Osteoclasts, chondrocytes, synovial cells, neutrophils/macrophages and fragmented particles of degraded extracellular matrix (which activate synovial cells and neutrophils to release ROS) are excellent sources of superoxide. Furthermore, ischemia-reperfusion takes place as the inflamed joint is used and favors the production of excess free radicals. Indeed, the mechanical function of the synovial joint distinguishes it from other tissues. It has been suggested that this mechanical activity and the continued use of an inflamed joint leads to the intermittent ischemia-reperfusion cycling which in turn results in pulses of radical activity in the joint leading to the chronicity of inflammation. As in many other organs, post-reperfusion release of O₂^{•-} in the ischemic organ plays a primary role in tissue damage.

Reactive oxygen species contribute significantly to tissue injury in RA and other inflammatory diseases. See Bauerova et al., “Role of Reactive Oxygen and Nitrogen Species in Etiopathogenesis of Rheumatiod Arthritis” Gen Physiol Biophys 1999 October; 18 Spec No.: 15-20. It is known, for example, that the superoxide anion is involved in the breakdown of proteins, lipids, DNA, uric acid, polysaccharides, which have been shown to be increased in rheumatoid arthritis patients. These proteins, lipids, DNA uric acid, and polysaccharides are protected from breakdown by superoxide dismutase. Also, ROS are directly involved in tissue injuries and indirectly facilitate tissue destruction by inactivating α-1 -protease inhibitors that form a complex with elastase, a serine proteinase. Bauerova et al., Role of Reactive Oxygen and Nitrogen Species in Etiopathogenesis of Rheumatoid Arthritis, Gen. Physiol. Biophys. 18, Focus Issue, 15-20 (1999). Studies have shown that chondrocyte-derived ROS damage cartilage matrix and mediate matrix degradation as part of the pathogenesis of both cartilage aging and osteoarthritis. Tiku et al., Evidence Linking Chondrocyte Lipid Peroxidation to Cartilage Matrix Protein Degradation, J. Biol. Chem., Vol. 275, No. 26, 20069-20076 (Jun. 30, 2000); Mattey et al., Influence of Polymorphism in the Manganese Superoxide Dismutase Locus on Disease Outcome in Rheumatoid Arthritis, Arthritis & Rheumatism, Vol. 43, No. 4, 859-864 (April 2000).

ROS have also been implicated in the damage of hyaluronic acid (“HA”), which is depolymerised causing synovial fluid to lose its lubricating properties causing friction in the joint. Kataoka et al., Hydroxyl radical scavenging activity of nonsteroidal antiinflammatory drugs, Free Radical Res. 27, 419-427 (1997). Hyaluronan attacked by ROS yields several intermediates and end-products found in increased concentrations in the synovial fluid and serum of rheumatic patients. Orvisky et al., High-molecular-weight hyaluronan a valuable tool in testing the antioxidative activity of amphiphilic drugs stobadine and vinpocetine, J. Pharm. Biomed. Anal. 16, 419-424 (1997); Mertens, et al., Study of eosinophil-endothelial adhesion, production of oxygen radicals and release of eosinophil cationic protein by peripheral blood eosinophils of patients with rheumatoid arthritis, Clinical and Experimental Allergy, Vol. 23, 868-873 (1993). This suggests a central role for activated oxygen species derived from superoxide in the pathogenesis of rhematoid arthritis. See, e.g., Bauerova et al., Role of Reactive Oxygen and Nitrogen Species in Etiopathogenesis of Rheumatoid Arthritis, Gen. Physiol. Biophys., 18, 15-20 (1999).

Thus, it follows that one therapeutic approach to treat inflammatory disease, autoimmune disease, e.g., RA, is to remove ROS. Superoxide anions are normally removed in biological systems by the formation of hydrogen peroxide and oxygen in the following reaction (hereafter referred to as dismutation):
O₂^{•-}+O₂^{•-}+2H⁺→+O₂+H₂O₂.

This reaction is catalyzed in vivo by the ubiquitous superoxide dismutase enzyme (“SOD”). This reaction is the subject for which the natural superoxide dismutase enzyme or a SOD mimetic will catalyze for the purposes of this invention. Native SOD activity has been found in articular cartilage, but levels of native SOD enzyme in synovial fluids of RA patients, and other inflammatory disease and autoimmune disease sufferers, are significantly lower than those found in normal synovial fluids. This reduced SOD activity may at least partially contribute to the pathological events associated with RA and suggests that endogenous SOD may play a role in protecting cartilage from oxidant mediated degradation. Under normal circumstances, formation of O₂^{•-} is kept under tight control by endogenous superoxide dismutase (“SOD”) enzymes which include: the Mn enzyme in mitochondria (“SOD2”) and the Cu/Zn enzyme present in the cytosol (“SOD1”) and extracellular surfaces (“SOD3”). However, in acute and chronic inflammation, the production of O₂^{•-} is increased at a rate that overwhelms the capacity of the endogenous SOD enzyme defense system to remove them.

An exogenous SOD, Orgoteine® (bovine CuZnSOD), was used in preliminary clinical trials in patients with various inflammatory and autoimmune disorders, including RA and OA. Orgotein® attenuates the release of free radicals in the synovial fluid of RA patients and has shown promising results as a therapeutic in patients with rheumatoid arthritis and osteoarthritis. For instance, in patients with active classical rheumatoid arthritis affecting the knee, intra-articular injections of Orgotein® ameliorated signs and symptoms as evidenced by: improved RA activity index (morning stiffness, flexion range, pain, walking time), decrease in the level of rheumatoid factor, reduced intake of rescue acetaminophen and overall improvement in physicians and patient global ratings. Clinical studies in patients with OA also revealed amelioration-with respect to signs and symptoms.

Despite encouraging clinical results, Orgotein® had to be removed from the market because of its origin (bovine) and the development of immune responses against Orgotein® in some individuals. Other issues associated with the use of native SOD enzymes as therapeutic agents include: solution instability, bell-shaped dose response curves, high susceptibility to proteolytic digestion and limited cellular/organ penetration.

Several non-peptidic catalysts which mimic this superoxide dismutating activity have been discovered. Recently, a class of non-peptidic, low-molecular weight compounds proven to possess a comparable catalytic activity and the high selectivity of the native superoxide dismutase (“SOD”) enzymes have been reported and the use of these compounds has been suggested for assessing a better therapeutic approach in diseases mediated by superoxide overproduction (Salvemini et al., Science 8, 304-306 (1999)). A particularly effective family of non-peptidic catalysts for the dismutation of superoxide consists of the manganese(II), manganese(III), iron(II) or iron(III) complexes of nitrogen-containing fifteen-membered macrocyclic ligands which catalyze the conversion of superoxide into oxygen and hydrogen peroxide, as described in U.S. Pat. Nos. 5,610,293, 5,637,578, 5,874,421, 5,976,498, 6,084,093, 6,180,620, 6,204,259, 6,214,817, 6,395,725, and 6,525,041, each of which are incorporated herein by reference in their entirety. See also, Weiss, R. H., et al., “Manganese(II)-Based Superoxide Dismutase Mimetics: Rational Drug Design of Artificial Enzymes”, Drugs of the Future 21: 383-389 (1996); and Riley, D. P., et al., “Rational Design of Synthetic Enzymes and Their Potential Utility as Human Pharmaceuticals” (1997) in CatTech, I, 41.

These mimics of superoxide dismutase have been shown to have a variety of therapeutic effects, including anti-inflammatory activity. See Weiss, R. H., et al., “Therapeutic Aspects of Manganese (II)-Based Superoxide Dismutase Mimics” In “Inorganic Chemistry in Medicine”,(Farrell, N., Ed.), Royal Society of Chemistry, in Press; Weiss, R. H., et al., “Manganese-Based Superoxide Dismutase Mimics: Design, Discovery and Pharmacologic Efficacies” (1995), In “The Oxygen Paradox” (Davies, K. J. A., and Ursini, F., Eds.) pp. 641-651, CLEUP University Press, Padova, Italy; Weiss, R. H., et al., J. Biol. Chem., 271: 26149 (1996); and Hardy, M. M., et al., J. Biol. Chem. 269: 18535-18540 (1994). Other non-peptidic catalysts which have been shown to have superoxide dismutating activity are complexes of porphyrins with iron and manganese cations.

Clinical trials and animal studies with natural, recombinant and modified superoxide dismutase enzymes have been completed or are ongoing to demonstrate the therapeutic efficacy of reducing superoxide levels in the disease states noted above. However, numerous problems have arisen with the use of the enzymes as potential therapeutic agents, including lack of oral activity, short half-lives in vivo, immunogenicity with nonhuman derived enzymes, and poor tissue distribution.

Thus, the need presently exists for effective compositions and methods for preventing and treating inflammatory disease states associated with the overproduction of ROS. Also, there is a need for compositions and methods for preventing and treating the inflammatory and non-inflammatory effects of rheumatoid arthritis associated with the overproduction of ROS.

### BRIEF SUMMARY OF THE INVENTION

Accordingly, it is an object of the invention to overcome these and other problems associated with the related art.

This invention provides a method for treating, preventing, inhibiting, or reversing inflammatory disease and autoimmune disease, preferably arthritis, more preferably rheumatoid arthritis, in a subject, the method comprising administering a subject in need thereof a therapeutically effective amount of a pentaaza-macrocyclic ligand complex represented by the following formula:

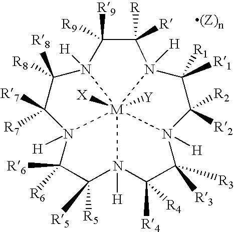

wherein R, R′, R₁, R′₁, R₂, R′₂, R₃, R′₃, R₄, R′₄, R′₅, R₆, R′₆, R₇, R′₇, R₈, R′₈, R₉ and R′₉ independently are selected from the group consisting of hydrogen and substituted or unsubstituted alkyl, alkenyl, alkynyl, cycloalkyl, cycloalkenyl, cycloalkylalkyl, cycloalkylcycloalkyl, alkylcycloalkyl, cycloalkenylalkyl, alkenylcycloalkyl, alkylcycloalkenyl, alkenylcycloalkenyl, heterocyclic, aryl and aralkyl radicals, or R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉, together with the carbon atoms to which they are attached independently form a substituted or unsubstituted saturated, partially saturated or unsaturated cyclic ring structure having 3 to 20 carbon atoms; or R or R′, R₁ or R′₁, and R₂ or R′₂, R₃ or R′₃ and R₄ or R′₄, R₅ or R′₅ and R₆ or R′₆, R₇ or R′₇, and R₈ or R′₈, and R₉ or R′₉, together with the carbon atoms to which they are attached independently form a substituted or unsubstituted nitrogen-containing heterocycle having 2 to 20 carbon atoms provided that when the nitrogen containing heterocycle is an aromatic heterocycle that does not have a hydrogen attached to the nitrogen, the hydrogen attached to the nitrogen in the macrocycle and the R groups attached to the same carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈ and R₉ and R′₉, together with the carbon atom to which they are attached independently form a substituted or unsubstituted saturated, partially saturated or unsaturated ring structure having 3 to 20 carbon atoms; or two of R, R′, R₁, R′₁, R₂, R′₂, R₃, R′₃, R₄, R′₄, R₅, R′₅, R₆, R′₆, R₇, R′₇, R₈, R′₈, R₉, and R′₉ attached to different carbon atoms of the macrocycle are bound to form a strap structure of the formula
—(CH₂)ₓ--M--(CH₂)_{w}--L--(CH₂)_{z}--J--(CH₂)_{y}—

wherein w, x, y and z independently are integers from 0 to 10 and M, L and J are independently selected from the group consisting of alkyl, alkenyl, alkynyl, cycloalkyl, aryl, heteroaryl, alkaryl, alkheteroaryl, aza, amido, ammonium, thio, sulfonyl, sulfinyl, sulfonamido, phosphonyl, phosphinyl, phosphino, phosphonium, keto, ester, carbamyl, ureido, thiocarbonyl, borate, borane, boraza, silyl, siloxy and silaza radicals, and combinations thereof; wherein X, Y and Z are pharmaceutically acceptable counterions or together are a pharmaceutically acceptable polydentate ligand, or are independently attached to one or more of the R groups and n is an integer from 0 to 3.

Preferably, the pentaaza-macrocyclic ligand complex is represented by the following formula:

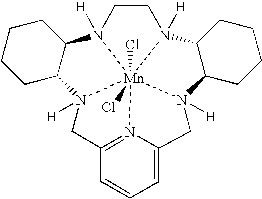

These and other features, aspects and advantages of the present invention will become better understood with reference to the following description, examples and appended claims.

### BRIEF DESCRIPTION OF THE SEVERAL VIEWS OF THE DRAWINGS

FIG. 1. Structure of M40403.

FIG. 2. Effect of M40403 on the onset of collagen-induced arthritis (“CIA”). The percentage of arthritic rats (rats showing clinical scores of arthritis>1) are represented (Graph 2A). Effect of M40403 (2-10 mg/kg i.p.) on the severity of collagen-induced arthritis. Median arthritic score during collagen-induced arthritis

FIG. 3. Comparing this figure (Graph 2B) to FIG. 2, there was a significant increase in the arthritic score from day 26 (P<0.01), and there was a significant suppression of the arthritic score way by M40403 between days 26 and 35 (P<0.01). Values are means±s.e. of 16 animals for each group. *p<0.01 versus Control. P<0.01 versus CIA

FIG. 4. Effect of M40403 (2-10 mg/kg i.p.) on CIA arthritis (secondary lesion). The swelling in hind paws over time (ml) was measured at 2 days intervals. Values are means±s.e. of 16 animals for each group. *p<0.01 versus Control. P<0.01 versus CIA

FIG. 5. Representative histology of the joint of a control animal (Picture 4A), an arthritic animal (Picture 4B in this figure and Picture 4D of FIG. 6 below), and an M40403 treated arthritic animal (Picture 4C of FIG. 6 below). Note the reduction in the degree of arthritis in the joint of the rat which was treated with M40403. Original magnification: A-B-C 100×; B140×. Photos is representative of at least 3 experiments performed on different experimental days.

FIG. 6. Depiction of arthritic animal as described in FIG. 5 above.

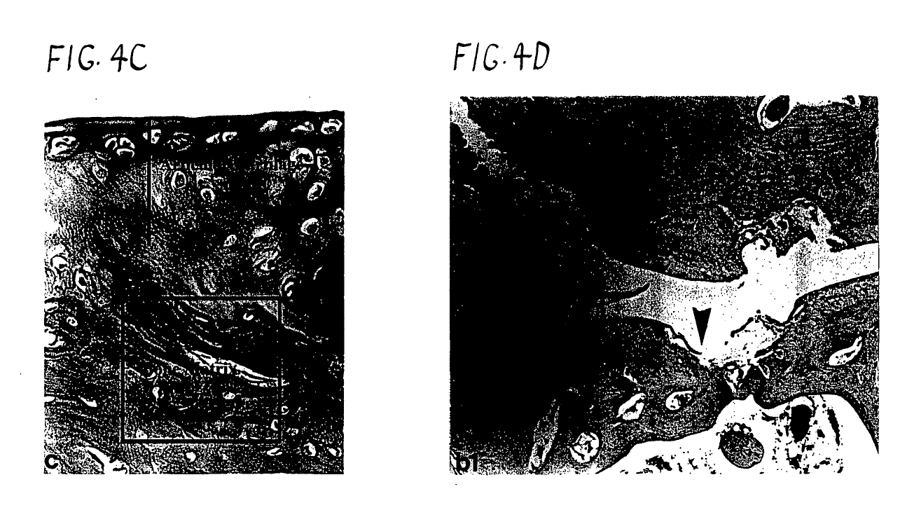

FIG. 7. Effect of M40403 treatments on histological damage score (Graph 5A), and radiograph score (Graph 5B). Values are means±s.e. of 16 animals for each group. *p<0.01 versus Control. P<0.01 versus CIA.

FIG. 8. Effect of M40403 treatments as described in FIG. 7 above.

FIG. 9. Radiographic progression of CIA in the tibiotarsal joint of rats with CIA. There is no evidence of pathology in the tibiotarsal joints of normal rats (Picture 6A). The hind paws from CII-immunized (35 days) rats demonstrated bone resorption (arrow) (Picture 6B). M40403 (5 mg/kg) suppressed joint pathology (arrow) and soft tissue swelling in the rat 5 hind paw (Picture 6C). Photos is representative of at least 3 experiments performed on different experimental days.

FIG. 10. Plasma levels of TNFa (Graph 7A). Cytokine levels were significantly reduced in the plasma from rats which received M40403 at 5 or 10 mg/kg. The dose of 2 mg/kg only attenuated the cytokines release. Values are means±s.e. means of 16 animals for each group. *p<0.01 versus sham. P<0.01 versus CIA

FIG. 11. Plasma levels of IL-1b (Graph 7B). Cytokine levels were significantly reduced in the plasma from rats which received M40403 at 5 or 10 mg/kg. The dose of 2 mg/kg only attenuated the cytokines release. Values are means±s.e. means of 16 animals for each group. *p<0.01 versus sham. P<0.01 versus CIA

FIG. 12. Nitrotyrosine immunostaining in the joint of a control rat (Picture 8A) and the paw of a rat at 35 days of collagen-induced arthritis (Picture 8B of this Figure and Picture 8B1 of FIG. 13 below). A marked increase in nitrotyrosine staining is evident in the joint in arthritis. There was a marked reduction in the immunostaining in the paw of rats which were treated with M40403 (5 mg/kg) (Picture 8C of FIG. 13 below). Original magnification: A-B-C 100×; B140×. Photos are representative of at least 3 experiments performed on different experimental days.

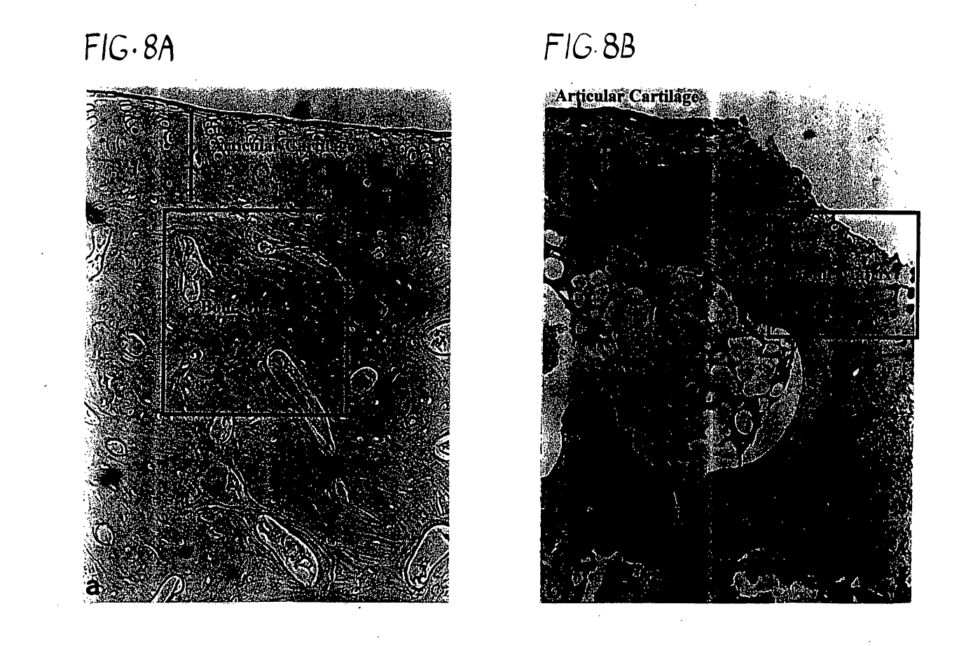

FIG. 13. Depiction of arthritic animal as described in FIG. 12 above.

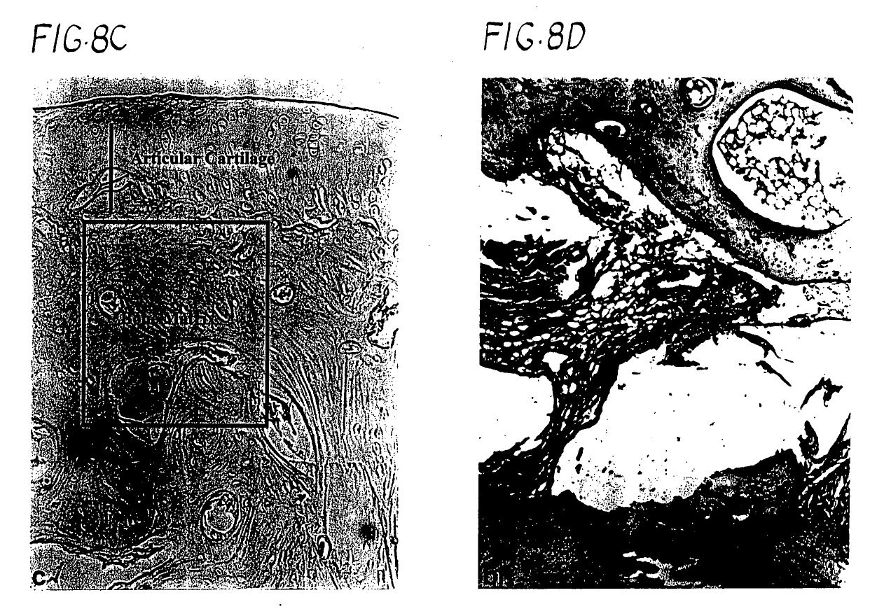

FIG. 14. Effect of M40403 on PARP activity: Staining was absent in control tissue (Picture 9A). 35 days following collagen-induced arthritis, PAR immunoreactivity was present in the joint from CII-immunized rats (Picture 9B of this Figure and Picture 9B1 of FIG. 15 below). In the paw of rats which received M40403 (5 mg/kg) (Picture 9C of FIG. 15 below), no positive staining was found. Original magnification: A-B-C 100×; B140×. Photos is representative of at least 3 experiments performed on different experimental days.

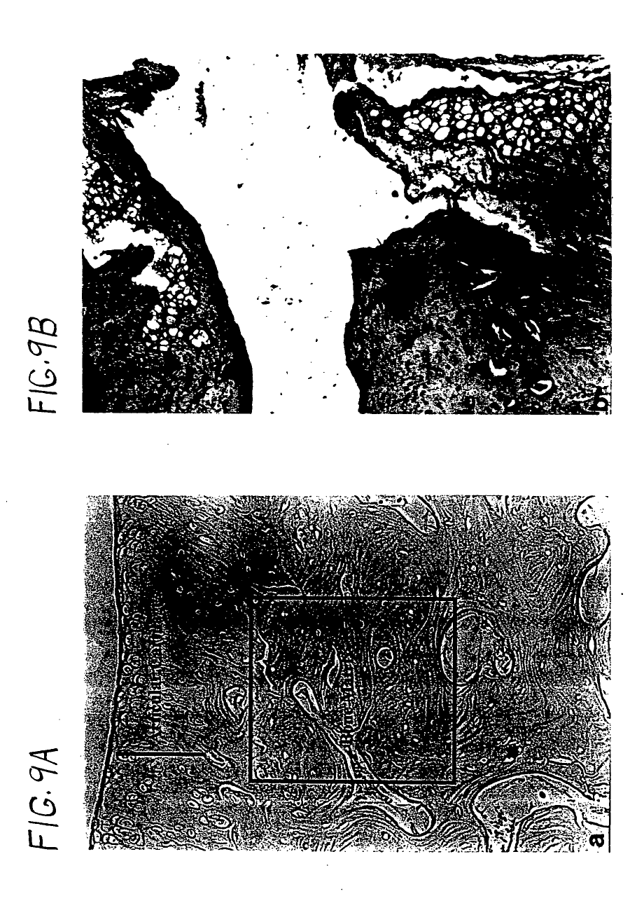

FIG. 15. Depiction of arthritic animal as described in FIG. 14 above.

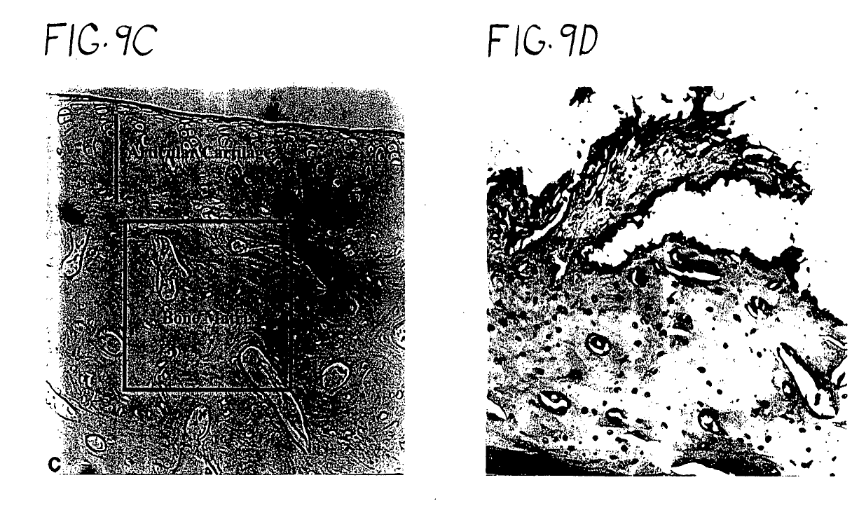

FIG. 16. Effect of M40403 on body weight gain. Beginning on day 25, the collagen-challenged rats gained significantly less weight than the normal rats, and this trend continued through day 35. M40403 (2-10 mg/kg) was able to positively affect the weight gain of CII-immunized rats. Values are means±s.e. means of 16 animals for each group. *p<0.01 versus Control. P<0.01 versus CIA.

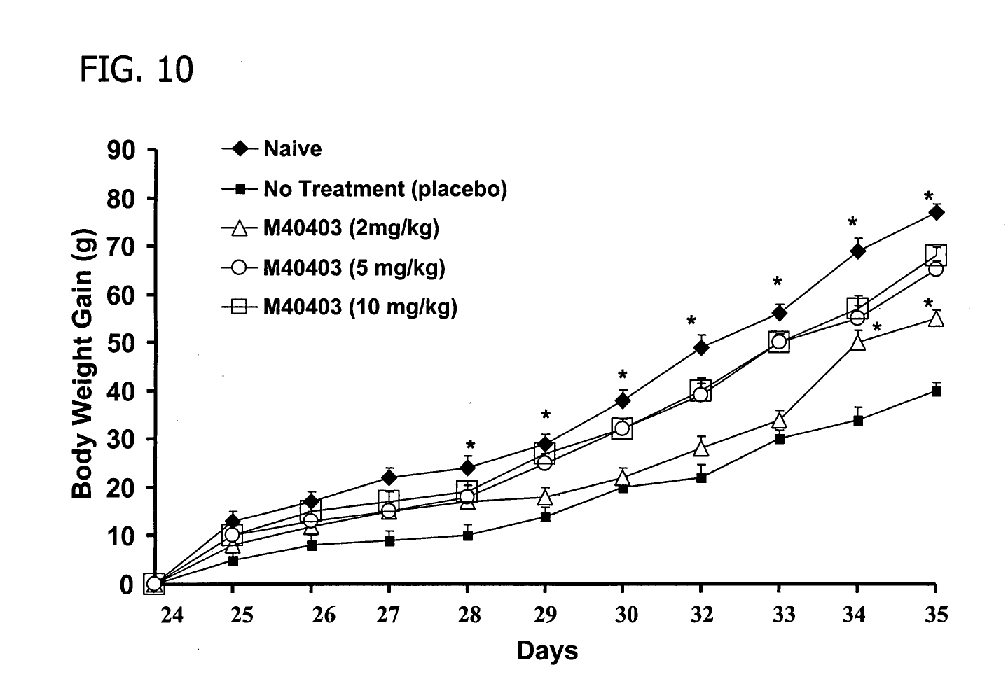

FIG. 17. Anti-CII antibody titers in rats with CIA. Serum was prepared from the blood of rats (day 35) treated daily with either vehicle or M40403. Values are means±s.e. means of 16 animals for each group. *p<0.01 versus Control.

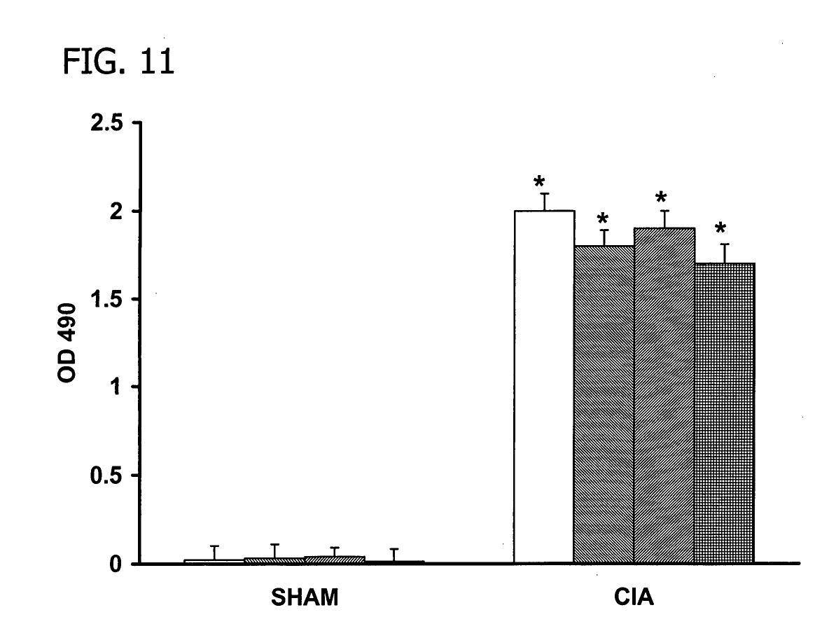

### DETAILED DESCRIPTION OF THE INVENTION

Abbreviations and Definitions

To facilitate understanding of the invention, a number of terms and abbreviations as used herein are defined below as follows:

As used herein, the terms “reactive oxygen species” or “ROS” refers to a toxic superoxide anion (O₂^{•-}). The superoxide anion, as well as the nitric oxide (NO^{•}) and the hydroxyl radical (OH^{•}), are different types of free-radicals.

As used herein, the terms “non-peptidic catalysts for the dismutation of superoxide” or “non-proteinaceous catalysts for the dismutation of superoxide” mean a low-molecular weight catalyst for the conversion of superoxide anions into hydrogen peroxide and molecular oxygen. These catalysts commonly consist of an organic ligand and a chelated transition metal ion, preferably copper, manganese(II), manganese(III), iron(II) or iron(III). The term may include catalysts containing short-chain polypeptides (under 15 amino acids) or macrocyclic structures derived from amino acids, as the organic ligand. The term explicitly excludes a superoxide dismutase enzyme obtained from any species.

The term “catalyst for the dismutation of superoxide” means any catalyst for the conversion of super oxide anions into hydrogen peroxide and molecular oxygen. The term explicitly includes a superoxide dismutase enzyme obtained from any species.

The mammal patient in the methods of the invention is a mammal suffering from inflammatory disease or disorder. It is envisioned that a mammal patient to which the catalyst for the dismutation of superoxide will be administered, in the methods or compositions of the invention, will be a human. However, other mammal patients in veterinary (e.g., companion pets and large veterinary animals) and other conceivable contexts are also contemplated.

As used herein, the terms “treatment” or “treating” relate to any treatment of inflammatory disease or disorders and include: (1) preventing inflammatory disease from occurring in a subject; (2) inhibiting the progression or initiation of the inflammatory disease, i.e., arresting or limiting its development; or (3) ameliorating or relieving the symptoms of the inflammatory disease.

As used herein, the terms “inflammatory disease” or “inflammatory disorder” refers to any disease marked by inflammation, which may be caused by a multitude of inciting events, including radiant, mechanical, chemical, infections, and immunological stimuli. Some inflammatory diseases include, but are not limited to, arthritis, inflammatory bowel disease, asthma, psoriasis, organ transplant rejections, radiation-induced injury, cancer, lupus and other autoimmune disorders, burns, trauma, stroke, rheumatic disorders, renal diseases, allergic diseases, infectious diseases, ocular diseases, skin diseases, gastrointestinal diseases, hepatic diseases, cerebral edema, sarcoidosis, thrombocytopenia, spinal cord injury, and autoimmune disorders.

As used herein, the term “arthritis” refers to inflammation of the joints and refers to a group of more than 100 rheumatic diseases that cause joint swelling, tissue damage, stiffness, pain (both acute and chronic), and fever. Arthritis can also affect other parts of the body other than joints including but not limited to: synovium, joint space, collagen, bone, tendon, muscle and cartilage, as well as some internal organs. The two most common forms of arthritis are osteoarthritis (“OA”) and rheumatoid arthritis (“RA”).

As used herein, the term “therapeutically effective amounts” means those amounts that, when administered to a particular subject in view of the nature and severity of that subject's disease or condition, will have the desired therapeutic effect, e.g., an amount which will cure, or at least partially arrest or inhibit the disease or condition.

As used herein, the term “joint” or “joints” refers to the place of union or junction between two or more bones of the skeleton.

SODm Therapy for Treatment, Prevention, Inhibition and Reversal of Inflammatory Disease

The present invention is directed to methods and compositions for the prevention and treatment of inflammatory diseases comprising administering preferred compositions containing a non-proteinaceous catalyst for dismutation of superoxide. The compositions of this invention may be administered to the subject subcutaneously, intravenously/or intramuscularly. In a preferred embodiment, the compositions of this invention are administered to a subject subcutaneously or intramuscularly.

Preferably, the compound employed in the method of the present invention will comprise a non-proteinaceous catalyst for the dismutation of superoxide anions (“SOD mimic”) as opposed to a native form of the SOD enzyme. As utilized herein, the term “SOD mimic” means a low-molecular-weight catalyst for the conversion of superoxide anions into hydrogen peroxide and molecular oxygen. These catalysts consist of an organic ligand having a pentaazacyclopentadecane portion and a chelated transition metal ion, preferably manganese or iron. The term may include catalysts containing short-chain polypeptides (under 15 amino acids), or macrocyclic structures derived from amino acids, as the organic ligand. The term explicitly excludes a SOD enzyme obtained from any natural sources. SOD mimics are useful in the method of the present invention as compared to native SOD because of the limitations associated with native SOD therapies such as, solution instability, limited cellular accessibility due to their size, immunogenicity, bell-shaped dose response curves, short half-lives, costs of production, and proteolytic digestion (Salvemini et al., (1999) Science 286: 304-306). For example, the best known native SOD, CuZn, has a molecular weight of 33,000 kD. Contrastingly, the instant SOD mimics have an approximate molecular weight of 400 to 600 Daltons.

In a preferred embodiment, the SOD mimics utilized in the present invention comprise an organic ligand chelated to a metal ion. A particularly preferred catalyst is a are pentaaza-macrocyclic ligand compound, more specifically a manganese chelate of a pentaazacyclopentadecane compound.

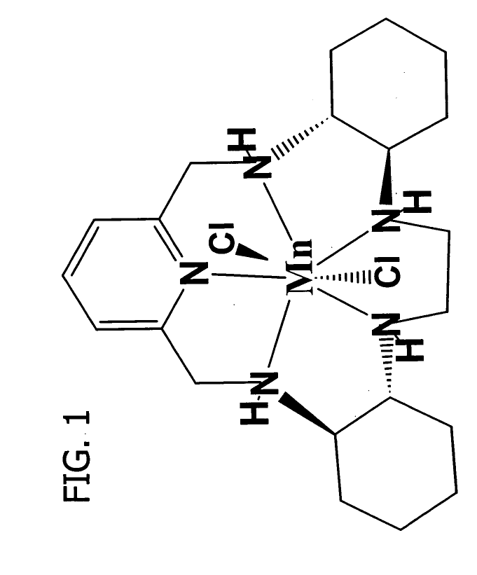

M40403 is a stable low molecular weight, manganese- containing, non-peptidic molecule possessing the function and catalytic rate of native SOD enzymes, but with the advantage of being a much smaller molecule with a molecular weight of 483 Daltons. M40403 is not only a highly active catalyst for the dismutation of O₂^{•-}, but it is also highly selective for superoxide. M40403 does not react with hydrogen peroxide, nor does it directly react with other biologically relevant oxidants such as nitric oxide or peroxynitrite. M40403 is represented in the alternative equivalent FIG. 1 and by the following formula:

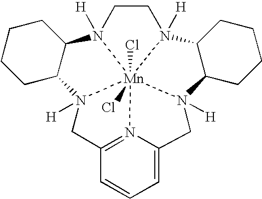

It has been discovered that M40403 is highly effective when used in the treatment of inflammatory disease and pain associated with inflammatory disease in a mammal. Particularly, M40403 demonstrates effectiveness when used in the treatment of arthritis, and more particularly, in the treatment of rheumatoid arthritis. The example below presents the results of experimentation with M40403 given intraperitoneally to subjects with collagen-induced arthritis (“CIA”). CIA is a model of experimental arthritis that is induced by the injection of type II collagen (“CII”). The similarities between the joint pathology in CIA and RA suggest that CIA is a relevant animal model useful in the search for new anti-arthritic drugs. The experiment of the example below demonstrates that M40403 is highly protective in a rat model of CIA. Surprisingly, it has been discovered that protective effects of M40403 were not limited to an overall anti-inflammatory effect but included significant protection of cartilage/bone compared to untreated collagen-immunized animals, as well as inhibition of key pro-inflammatory cytokines known to be involved in the human disease.

Activity of the complexes of the present invention for catalyzing the dismutation of superoxide can be demonstrated using the stopped-flow kinetic analysis technique as described in Riley, D. P. et al., Anal. Biochem., 196: 344-349 (1991) which is incorporated herein by reference. The stopped-flow kinetic analysis is suitable for screening compounds for SOD activity or complexes of the present invention, as shown by stopped-flow analysis, correlate to treating the above disease states and disorders. However, the stopped-flow analysis is not an appropriate method for demonstrating the activity of all superoxide dismutase mimics. Other methods may be appropriate or preferred for some SOD mimics. See Weiss et al., Evaluation of Activity of Putative Superoxide Dismutase Mimics. Direct Analysis by Stopped-flow Kinetics, J. Biol. Chem. 268 (31): 23049-54 (Nov. 5, 1993).

For use in treatment or prophylaxis of subjects, the compounds of the invention can be formulated as pharmaceutical or veterinary compositions. Depending on the subject to be treated, the mode of administration, and the type of treatment desired (e.g., inhibition, prevention, prophylaxis, therapy), the compounds are formulated in ways consonant with these parameters. The compositions of the present invention comprise a therapeutically or prophylactically effective dosage of a catalyst for the dismutation of superoxide in combination with at least one corticosteroid. The catalyst for the dismutation of superoxide is preferably a SOD mimetic, as described in more detail above. More preferably, the SOD mimetic is compound M40403. The SODms of this invention are preferably used in combination with a pharmaceutically acceptable carrier, either in the same formulation or in separate formulations.

The compositions of the present invention may be incorporated in conventional pharmaceutical formulations (e.g. injectable solutions) for use in treating humans or animals in need thereof. Pharmaceutical compositions can be administered by subcutaneous, intravenous, or intramuscular injection, or as large volume parenteral solutions and the like. The term parenteral as used herein includes subcutaneous injections, intravenous, intramuscular, intrasternal injection, or infusion techniques.

For example, a parenteral therapeutic composition may comprise a sterile isotonic saline solution containing between 0.1 percent and 90 percent weight to volume of the catalysts for the dismutation of superoxide. A preferred solution contains from about 5 percent to about 25 weight percent catalysts for dismutation of superoxide in solution (% weight per volume).

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the

Total daily dose administered to a subject in single or divided doses may be in amounts, for example, from about 0.00025 to about 20 mg/kg body weight daily, more preferably from about 0.001 to about 10 mg/kg body weight daily, and more usually about 0.01 to about 3 mg/kg body weight daily, when given as a parenteral injection or continuous infusion.

Dosage unit compositions may contain such amounts of sub-multiples thereof to make up the daily dose. The amount of active ingredient that may be combined with the carrier materials to produce a single dosage form will vary depending upon the subject treated and the particular mode of administration. For instance, systems such as transdermal administration or oral administration, which are substantially less efficient delivery systems, may require dosages at least an order of magnitude above those required for parenteral administration. The amount of active ingredient that may be combined with the carrier materials to produce a single dosage form will vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regimen for treating a disease condition with the compounds and/or compositions of this invention is selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely and therefore may deviate from the preferred dosage regimen set forth above.

The pharmaceutical compositions of the present invention are preferably administered to a human. However, besides being useful for human treatment, these extracts are also useful for veterinary treatment of companion animals, exotic animals and farm animals, including mammals, rodents, avians, and the like. More preferred animals include horses, dogs, cats, sheep, and pigs.

The detailed description set-forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

Superoxide Dismutase Mimetics

This invention is based upon surprising discoveries involving certain organometallic complexes designed as synthetic catalysts for use in the body. These catalysts have been designed as synthetic replacements for or adjuncts to the naturally occurring enzyme superoxide dismutase (SOD).

Naturally occurring SOD scavenges and eliminates the toxicity of free superoxide radicals (O₂^{-•}) liberated by certain metabolic reactions. Although these free radicals play a major (and deleterious) role in the inflammatory response and other toxic reactions to injury, neither superoxide nor SOD has been known to be directly involved in pain perception. In addition, SOD has a very short biological half-life, on the order of seconds or minutes rather than hours, so it would be considered unsuitable for treatment of conditions in which increased dismutation of superoxide radicals would be desirable over periods of from minutes to days.

Dismutation of superoxide radicals is catalyzed by a coordinated transition metal ion. In the natural SOD enzyme, the metal is manganese, copper or zinc and the coordination complex is a conventional protein structure. Synthetic SOD catalysts also use transition metals, complexed with low molecular weight organic ligands, generally polydentate N-containing macrocycles. These molecules have been designed to be highly efficient and to overcome the pharmacokinetic disadvantages of natural SOD enzyme. The k_{cat} of some of these compounds is as high as about 10⁹ (see Example 170), indicating extraordinary catalytic efficiency, as effective as the natural enzyme and approaching the theoretical rate at which diffusion can deliver free radical substrate to the catalyst under biological conditions. They also have oil:water partition coefficients (_{log}P) that provide excellent bioavailability, and stability in the body on the order of hours to days. Their small size and low molecular weight makes it possible for the synthetic catalysts to cross membrane barriers that restrict movement of natural SOD, and their non-protein structure reduces the risk of allergic reactions that have been a problem with the administration of protein-based recombinant SOD. Finally, natural SOD produces hydrogen peroxide in the process of dismutating superoxide, yet hydrogen peroxide inhibits natural SOD, effectively self-limiting the efficacy of the natural compound. In contrast, synthetic small-molecule SOD catalysts are not susceptible to the action of hydrogen peroxide and thus retain their effectiveness.

In combination with the discovery that synthetic SOD catalysts are highly effective as analgesics to prevent or provide relief from pain in conditions in which the pain threshold is elevated, it is now shown that the pentaaza macrocyclic complexes of the present invention enhance the ability to treat, prevent, inhibit and reverse inflammatory disease.

No known mechanism accounts for the analgesic properties of these compounds. However, the data shown in the examples illustrate that these compounds can be as effective as morphine in preventing and relieving certain kinds of pain. Y. Lin et al., Int. J. Maxillofac. Surg. 23:428-429 (1994) reported the use of intra-articular injections of human Cu/Zn superoxide dismutase as a nonsteroidal anti-inflammatory in the treatment of temporomandibular joint dysfunction. Positive response in terms of mandibular movement and pain was observed in 83% of patients. The authors note that the results are remarkable because SOD has been studied and shown to exert no peripheral or central analgesic effect. They attribute the reduction in pain to the reduction in tissue injury and inflammation associated with TMJ dysfunction.

Similarly, no known mechanism accounts for the ability of these compounds to prevent or reverse tolerance to opioids. G. I. Elmer et al., Euro. J. Pharmacol. 283 (1995) 227-232, reported that transgenic mice expressing the human Cu/Zn superoxide dismutase gene had an increase in μ-opioid receptor concentration in dopaminergic related tissues and the central grey area of the CNS, which was associated with a dose-related increased sensitivity to μ-receptor agonists such as morphine. At the same time the authors also observed conflicting effects of transgenic SOD on δ-receptor agonists (mice heterozygous for the transgene were more sensitive than homozygotes, which were more sensitive than untransformed mice) and observed no effect of transgenic SOD on κ-receptor agonists.

Superoxide dismutase activity is known to play a critical role in regulating the redox state of the cell, as reported by J. L. Cadet, Int. J. Neurosci. 40, 13 (1988). This in turn is reported by Marzullo and Hine, Science 208, 1171 (1980) to significantly affect in vitro μ-and δ-opioid binding.

In particular, this invention provides a method of producing analgesia in a human or lower mammal patient, comprising administering to the patient an analgesic amount of a functional synthetic catalyst for the dismutation of superoxide radicals. Based on the data obtained, it is reasonable to expect that any superoxide dismutase catalyst will be effective in the practice of this invention. A preferred synthetic catalyst is a coordination complex of transition metal with an organic ligand. Preferred transition metals are copper, manganese and zinc. Manganese is most preferred. In general, the organic ligand is a N-containing macrocycle, and most preferred complexed ligands are selected from the group consisting of compounds of the formula:

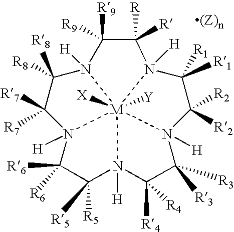

wherein R, R′, R₁, R′₁, R₂, R′₂, R₃, R′₃, R₄, R′₄, R′₅, R₆, R′₆, R₇, R′₇, R₈, R′₈, R₉ and R′₉ independently are selected from the group consisting of hydrogen and substituted or unsubstituted alkyl, alkenyl, alkynyl, cycloalkyl, cycloalkenyl, cycloalkylalkyl, cycloalkylcycloalkyl, alkylcycloalkyl, cycloalkenylalkyl, alkenylcycloalkyl, alkylcycloalkenyl, alkenylcycloalkenyl, heterocyclic, aryl and aralkyl radicals, or R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉, together with the carbon atoms to which they are attached independently form a substituted or unsubstituted saturated, partially saturated or unsaturated cyclic ring structure having 3 to 20 carbon atoms; or R or R′, R₁ or R′₁, and R₂ or R′₂, R₃ or R′₃ and R₄ or R′₄, R₅ or R′₅ and R₆ or R′₆, R₇ or R′₇, and R₈ or R′₈, and R₉ or R′₉, together with the carbon atoms to which they are attached independently form a substituted or unsubstituted nitrogen-containing heterocycle having 2 to 20 carbon atoms provided that when the nitrogen containing heterocycle is an aromatic heterocycle that does not have a hydrogen attached to the nitrogen, the hydrogen attached to the nitrogen in the macrocycle and the R groups attached to the same carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈ and R₉ and R′₉, together with the carbon atom to which they are attached independently form a substituted or unsubstituted saturated, partially saturated or unsaturated ring structure having 3 to 20 carbon atoms; or two of R, R′, R₁, R′₁, R₂, R′₂, R₃, R′₃, R₄, R′₄, R₅, R′₅, R₆, R′₆, R₇, R′₇, R₈, R′₈, R₉, and R′₉ attached to different carbon atoms of the macrocycle are bound to form a strap structure of the formula
—(CH₂)ₓ--M--(CH₂)_{w}--L--(CH₂)_{z}--J--(CH₂)_{y}—

wherein w, x, y and z independently are integers from 0 to 10 and M, L and J are independently selected from the group consisting of alkyl, alkenyl, alkynyl, cycloalkyl, aryl, heteroaryl, alkaryl, alkheteroaryl, aza, amido, ammonium, thio, sulfonyl, sulfinyl, sulfonamido, phosphonyl, phosphinyl, phosphino, phosphonium, keto, ester, carbamyl, ureido, thiocarbonyl, borate, borane, boraza, silyl, siloxy and silaza radicals, and combinations thereof; wherein X, Y and Z are pharmaceutically acceptable counterions or together are a pharmaceutically acceptable polydentate ligand, or are independently attached to one or more of the R groups and n is an integer from 0 to 3.

Surprisingly, such compositions are also able to enhance the ability to treat, prevent, inhibit and reverse inflammatory disease. By an “analgesic amount” and “therapeutically effective amount” of the synthetic SOD catalysts herein is meant an amount that significantly prevents or alleviates pain in the human or lower animal being treated. At a certain level stimuli are perceived as painful, while below that level they are not. This level is referred to as the pain threshold. Healthy, normal subjects exhibit a normal pain threshold that can be quantified for a given stimulus. A normal healthy individual perceives a pin prick as painful, but does not perceive the movement of a joint within its normal range of motion as painful. An individual suffering from arthritis has a lowered pain threshold and will perceive such normal movement as painful. An individual suffering from sunburn has a lowered pain threshold and may perceive the touch of a finger to be as painful as a normal individual perceives a pin prick. Because these compounds operate to elevate a lowered pain threshold, they will be effective in the treatment of such pain, and an “analgesic amount” of synthetic SOD catalysts in the treatment methods provided here also means an amount that significantly elevates the pain threshold above its pre-treatment level or prevents the pain threshold from being lowered by a pathological condition. From the standpoint of the pharmacologist and pharmaceutical scientist, this can be measured prospectively using common animal models such as the phenylquinone writhing model, the rat tail flick (radiant heat) model, the carrageenan inflammation model, the Freund's adjuvant model, and other pain models well known to pharmacological science. From the standpoint of the clinician, this can be measured according to the subjective response of each patient to a unit dose of the compound, and subsequent doses can be titrated to achieve the desired level of analgesia within the therapeutic range of the compound employed.

By an “amount sufficient to prevent or reverse tolerance to opioids” is meant The dual administration of a superoxide dismutase catalyst together with an opioid such as morphine or fentanyl allows lower doses of the morphine or fentanyl to elicit its analgesic effects while limiting its side effects. Moreover, a superoxide dismutase catalyst can reverse opioid tolerance in patients who have already developed tolerance. Thus, the superoxide dismutase catalysts restore the analgesic effect lost during prolonged treatment with an opioid. These catalysts prevent or reverse the tolerance to opioids without many of the side effects of other compounds proposed for this purpose, such as clonidine and buprenorphine. And in contrast to other proposed compounds, such as inhibitors of inducible nitric oxide synthase, the superoxide dismutase catalysts themselves have potent analgesic effects that are useful in hyperalgesic conditions such as burns, arthritis and other inflammatory diseases, migraine, and pain associated with tumor infiltration and cancer therapy.

The compounds of this invention are also useful as adjuncts in the prevention and treatment of pain with opioid analgesics, nitric oxide donors or nonsteroidal anti-inflammatory compounds. In preferred embodiments, the superoxide dismutase catalyst is administered conjointly with the opioid, NO₂ donor or NSAID compound. Administered in conjunction with an opioid, the superoxide dismutase catalyst potentiates the opioid and prevents development of tolerance and hyperalgesia. Administered after opioid tolerance, hyperalgesia and/or dependency have developed, the superoxide dismutase catalyst reverses the tolerance and hyperalgesia and reduces the symptoms of the withdrawal syndrome. Administered in conjunction with an NSAID compound or nitric oxide donor, the superoxide dismutase catalyst potentiates both the analgesia and the inflammatory action of the NSAID or NO₂ donor. These drug moieties can also be linked to provide bifunctional compounds of the formula Aₙ-Qₘ, wherein A is a superoxide dismutase catalyst moiety, Q is selected from nonsteroidal anti-inflammatory drug moieties, nitric oxide donor moieties and opioid analgesic drug moieties, and n and m are independently integers from 1 to 3. Depending upon the selection of A and Q, this can easily be done by substituting the NSAID or opioid moiety for one or more of counterion/ligands X, Y and Z in the preferred formula above. A simple approach to providing a combination containing a nitric oxide donor is to attach one or more nitrate or nitrite groups to the superoxide dismutase compound.

While not intending to be limited by theory, it is believed that the opioid withdrawal syndrome has many symptoms in common with the withdrawal syndromes associated with other addictive compounds and behaviors, including symptoms of withdrawal from cocaine, nicotine, and eating disorders such as anorexia and bulimia, especially the hyperreflexia and hyperalgesia associated with withdrawal. Accordingly, this invention also provides a method of preventing and treating symptoms of addition withdrawal, by administering to a patient in need of such treatment an amount of a superoxide dismutase catalyst that is safe and effective to prevent or reduce such symptoms.

A safe and effective amount of the compounds used in the practice of this invention is an amount that provides analgesia, thereby alleviating or preventing the pain being treated at a reasonable benefit/risk ratio as is intended with any medical treatment. In using the compounds for the reversal of opioid tolerance or reduction of withdrawal symptoms, these endpoints are used rather than analgesia. Obviously, the amount of catalyst used will vary with such factors as the particular condition that is being treated, the severity of the condition, the duration of the treatment, the physical condition of the patient, the nature of concurrent therapy (if any), the route of administration, the specific formulation and carrier employed, and the solubility and concentration of catalyst therein.

By “systemic administration” is meant the introduction of the catalyst or composition containing the catalyst into the tissues of the body, other than by topical application. Systemic administration thus includes, without limitation, oral and parenteral administration.

Depending upon the particular route of administration, and compatibility with the active compound chosen, a variety of pharmaceutically-acceptable carriers, well-known in the art, may be used. These include solid or liquid filler, diluents, hydrotropes, excipients, surface-active agents, and encapsulating substances. The amount of the carrier employed in conjunction with the catalyst is sufficient to provide a practical quantity of material per unit dose.

Pharmaceutically-acceptable carriers for systemic administration that may be incorporated into the compositions of this invention, include sugars, starches, cellulose and its derivatives, malt, gelatin, talc, calcium sulfate, vegetable oil, synthetic oils, polyols, alginic acid, phosphate buffer solutions, emulsifiers, isotonic saline, and pyrogen-free water.

The catalysts can be administered parenterally in combination with a pharmaceutically acceptable carrier such as corn oil, Cremophor EL or sterile, pyrogen-free water and a water-miscible solvent (e.g., ethyl alcohol) at a practical amount of the catalyst per dose. Preferably, the pharmaceutically-acceptable carrier, in compositions for parenteral administration, comprises at least about 90% by weight of the total composition. Parenteral administration can be by subcutaneous, intradermal, intramuscular, intrathecal, intraarticular or intravenous injection. The dosage by these modes of administration is usually in the range of from about 0.1 mg to about 20 mg per day.

Various oral dosage forms can be used, including such solid forms as tablets, capsules, granules and bulk powders. These oral forms comprise a safe and effective amount, usually at least about 5%, and preferably from about 25% to about 50% of the catalyst. Tablets can be compressed, tablet triturates, enteric-coated, sugar-coated, film-coated or multiple compressed, containing suitable binders, lubricants, diluents, disintegrating agents, coloring agents, flavoring agents, preservatives, flow-inducing agents, and melting agents. Liquid oral dosage forms include aqueous solutions, emulsions, suspensions, solutions and/or suspensions reconstituted from noneffervescent granules and effervescent preparations reconstituted from effervescent granules, containing suitable solvents, preservatives, emulsifying agents, suspending agents, diluents, sweeteners, melting agents, coloring agents, and flavoring agents. Preferred carriers for oral administration include gelatin, propylene glycol, ethyl oleate, cottonseed oil and sesame oil. Specific examples of pharmaceutically-acceptable carriers and excipients that may be used to formulate oral dosage forms containing the catalysts used in this invention, are described in U.S. Pat. No. 3,903,297, Robert, issued Sep. 2, 1975, incorporated by reference herein. Techniques and compositions for making solid oral dosage forms are described in Marshall, “Solid Oral Dosage Forms,” Modern Pharmaceutics, Vol. 7 (Banker and Rhodes, editors), 359-427 (1979), incorporated by reference herein.

By “pharmaceutically acceptable salts” is meant those salts that are safe for topical or systemic administration. These salts include the sodium, potassium, calcium, magnesium, and ammonium salts.

Effect of M40403 in the Development of Collagen-Induced Arthritis

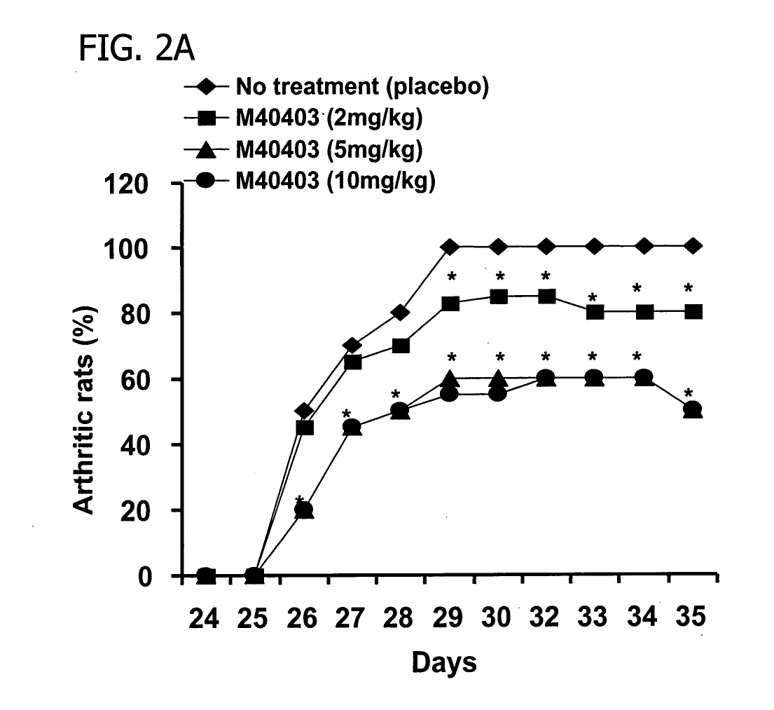

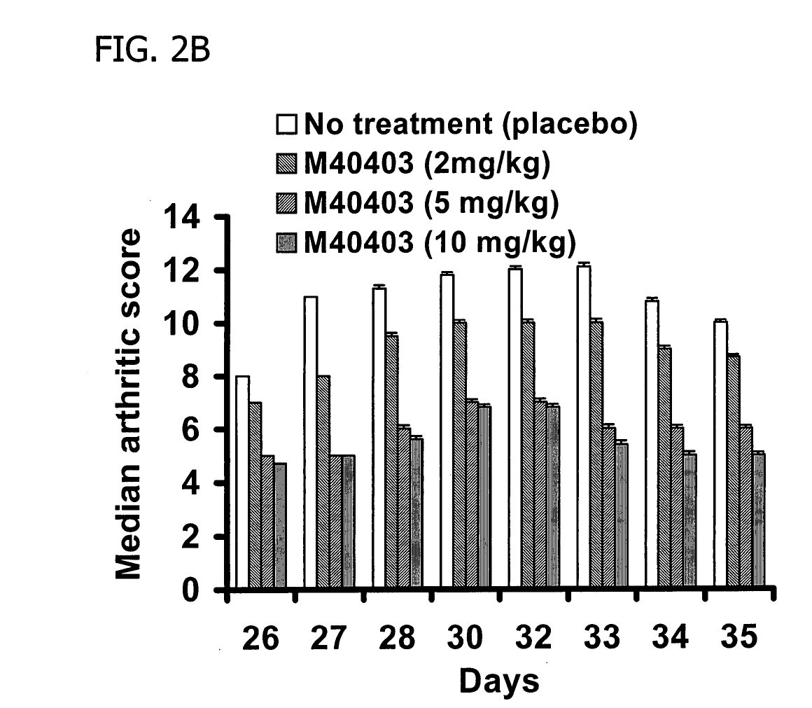

CIA developed in rats immunized with CII and clinical signs (periarticular erythema and edema) of the disease (FIG. 2A) first appeared in the hind paws between 24 and 26 after the first injection and consisted of mild erythema and swelling of the feet and ankles. Furthermore, a 100% incidence of CIA was observed by day 27 in-CII-immunized rats. In contrast the maximum incidence of CIA in rats which received M40403 at 5 or 10 mg/kg starting on day 25 was 50%, (FIG. 2A) (p<0.01). No significant difference was found between the two higher doses (5 and 10 mg/kg). Hind paw erythema and swelling increased in frequency and severity in a time-dependent mode with maximum arthritis indices of approximately 13 observed between 28 and 35 days post-immunization (FIG. 2B). M40403 attenuated (P<0.01) arthritis index score as observed between days 26 and 35 post-CII immunization (FIG. 2B). The data in FIG. 3 demonstrate a time-dependent increase in hind paw volume (ml, each value represents the mean values of both hind paws) in rats immunized with CII. Maximum paw volume occurred by day 35 in the CII-immunized rats. M40403 attenuated (P<001) hind paw swelling from day 26 and 35 post-immunization, achieving a maximal response of 56% from day 28 to 35 (FIG. 3). No significant difference was found between the two higher doses (5 and 10 mg/kg).

Effects of M40403 on CIA Histopathology and Radiographic Analysis of CIA

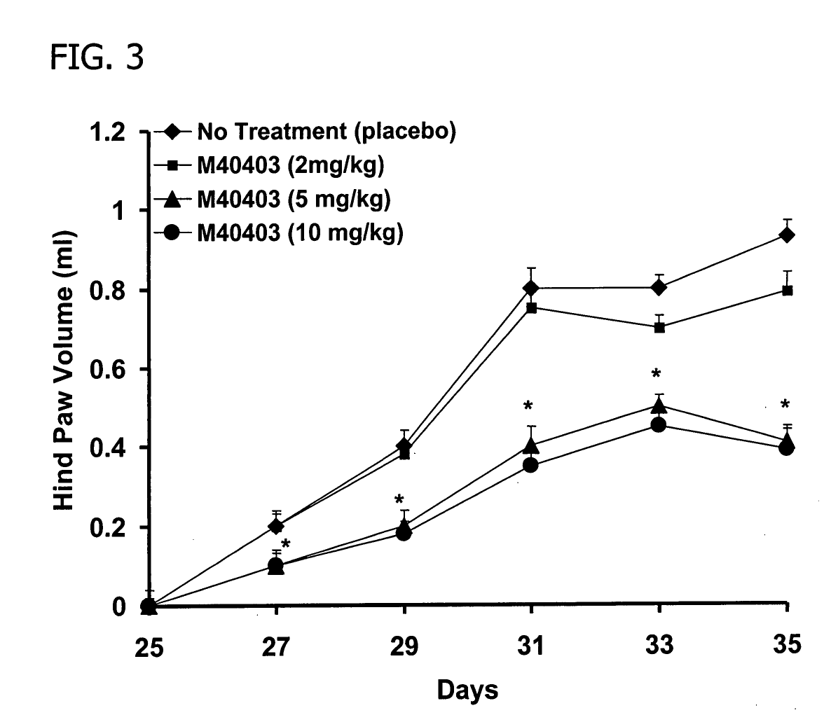

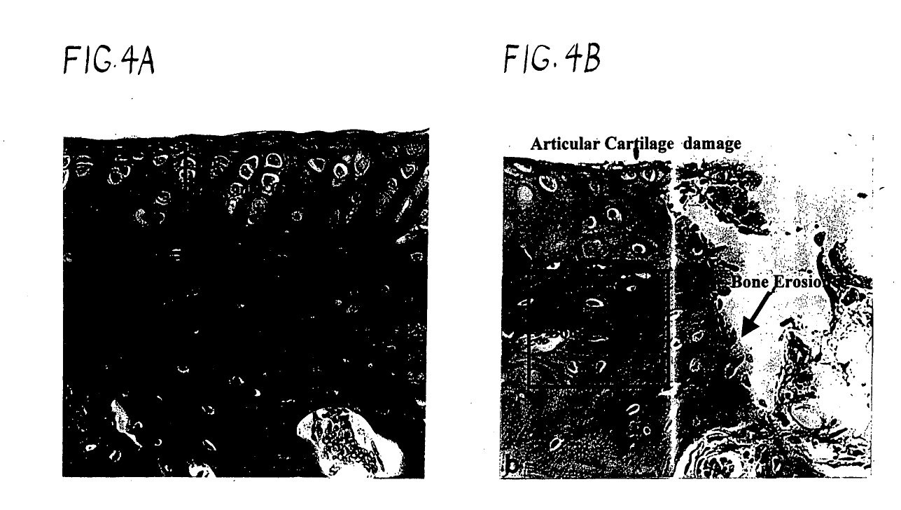

At day 35, histological evaluation of the joints in the vehicle-treated arthritic animals revealed signs of severe arthritis (FIG. 5A) characterized by articular cartilage and bone erosion (see small arrow FIG. 4B, B1, Table 1) as well as a massive inflammatory cells infiltration (see arrow, FIG. 4B1). In the animals which received M40403 (5 mg/kg), the degree of arthritis was significantly reduced: a moderate infiltration into several of the larger joints comprised primarily of neutrophils, coupled with mild articular cartilage and bone erosion, was observed (FIG. 4C, 5A, Table 1). A radiographic examination of hind paws from vehicle-treated rats 35 days post CII immunization revealed bone matrix resorption (FIG. 5B, 6B) in the tibiotarsal joint. In the proximal tibia the Ob.S/Bs, the ES/Bs and Oc.S/Bs were significantly increased at 35 days after CII immunization (Table 1). M40403 at 5 mg/kg markedly protected against bone resorption (FIG: 5B, 6C, Table 1). A similar protective effect was observed in the group of animals treated with M40403 at 10 mg/kg (FIG. 5). There was no evidence of pathology in naive rats (FIG. 4A, 5A, 6A, Table 1) .

TABLE 1

| | Ob.S/BS (%) | ES/BS (%) | Oc.S/BS (%) |
|---|---|---|---|
| Sham + Vehicle | 1.21 ± 1.32 | 26.66 ± 3.32 | 1.76 ± 1.52 |
| CIA + Vehicle | 9 ± 1.02* | 40.22 ± 2.12* | 8.32 ± 1.72* |
| CIA + M40403 | 3.1 ± 0.94 | 29.98 ± 4.1° | 3.21 ± 0.99° |
| (5 mg/kg) | | | |
| CIA + M40403 | 2.9 ± 1 | 28.42 ± 3.9° | 3.41 ± 1.02° |
| (10 mg/kg) | | | |

Data are expressed as the mean value s.e.
*p < 0.01 vs. sham;
°p < 0.01 vs. CIA.
Key:
OB.S/BS osteoblast surface;
ES/BS eroded surface;
Oc.S/Bs osteoclast surface.

Effect of M40403 on Cytokine Production

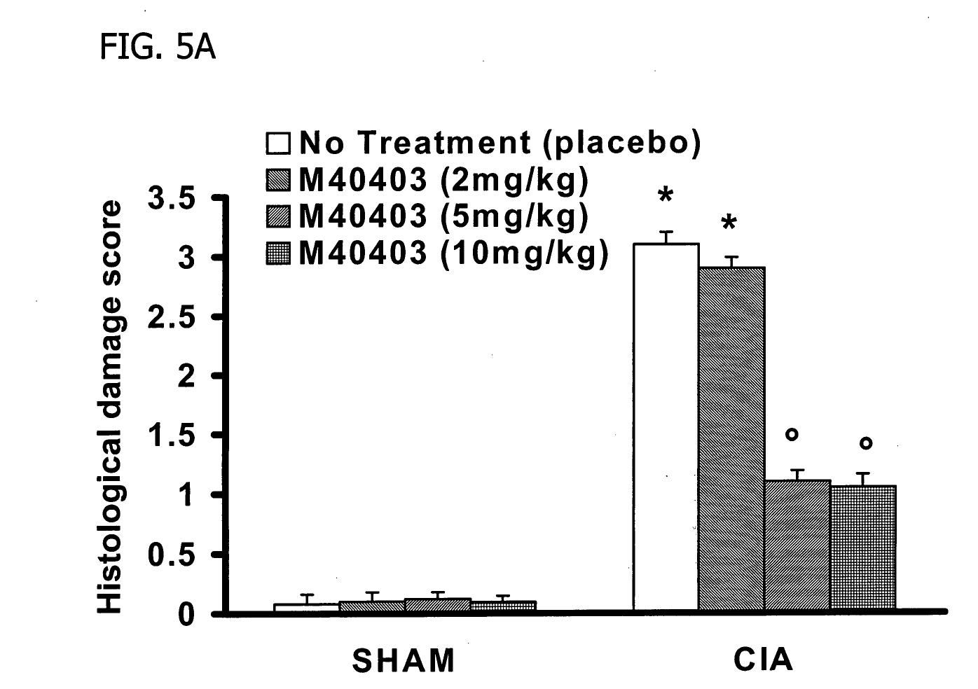

At day 35, the levels of TNFa and IL-1b were significantly elevated in the plasma of vehicle-treated CIA-immunized rats (FIG. 7). In contrast, the levels of these cytokines were significantly lower in rats which received M40403 at 5 or 10 mg/kg (FIG. 7). No significant difference was found between the two higher doses (5 and 10 mg/kg).

Nitrotyrosine Formation and PARP Activation

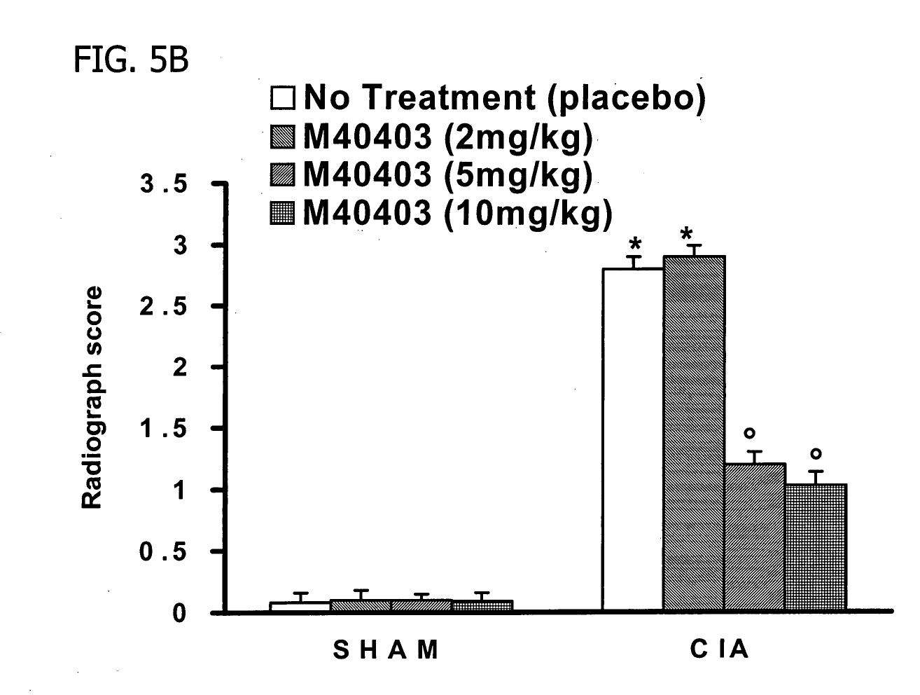

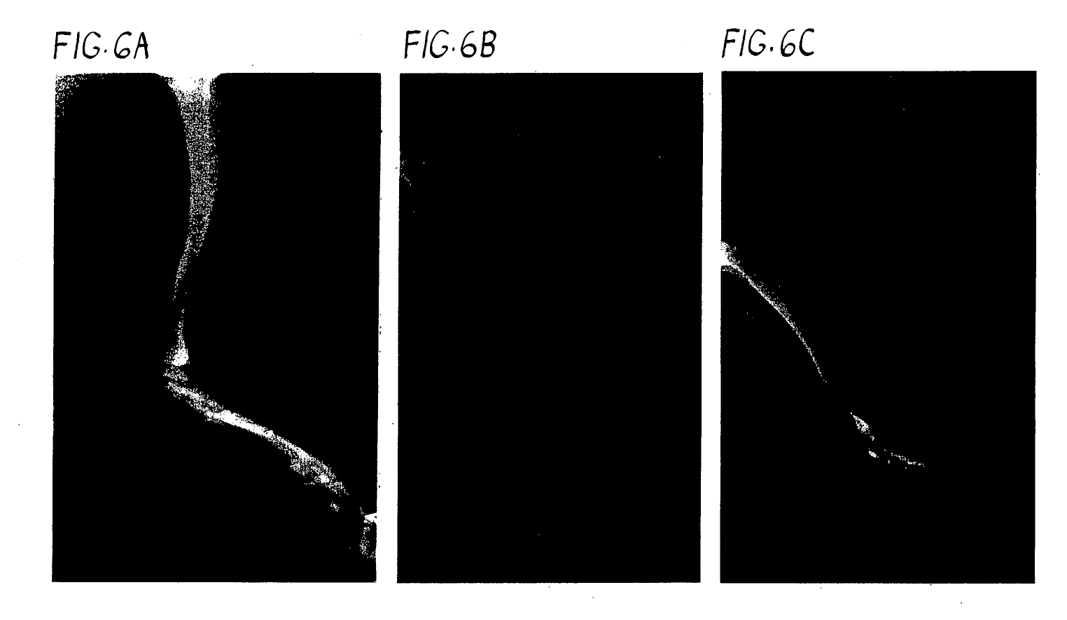

When compared to control groups (FIG. 8A), immunohistochemical analysis of joint sections obtained from vehicle-treated rats immunized with collagen type II revealed a positive staining (see arrows) for nitrotyrosine, which was primarily localized into articular cartilage and in damaged bone (FIG. 8B, B1). In contrast, no positive nitrotyrosine staining was found in the joints of CIA-immunized rats which had been treated with M40403 (5 mg/kg) (FIG. 8C). Immunohistochemical analysis of joint obtained from rats immunized with collagen type II also revealed a positive staining for PAR into articular cartilage and in damaged bone (FIG. 9B). In contrast, no positive staining for PAR was found in the joint of CIA-immunized rats which had been treated with M40403 (5 mg/kg) (FIG. 9C). There was no staining for either nitrotyrosine or PAR in joints obtained from naive rats (FIGS. 8A, 9A). Similar protective effect was observed in the group of animals treated with M40403 at 10 mg/kg (data not shown).

Effect of M40403 on Body Weight Gain

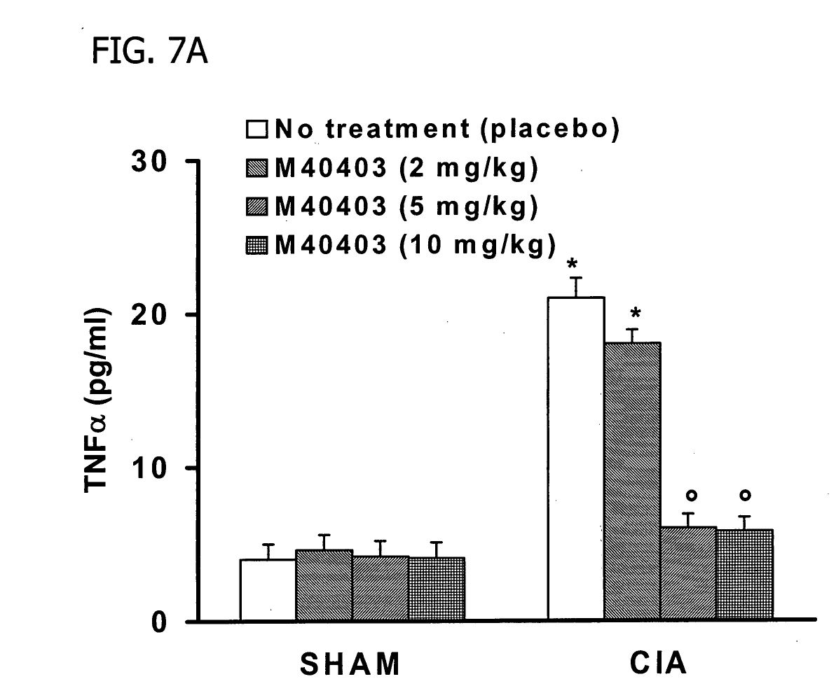

The rate and the absolute gain in body weight were comparable in naive rats and CII-immunized rats for the first week (FIG. 10). Beginning on day 25, the untreated collagen-immunized rats gained significantly less weight than the naive ones, and this trend continued through day 35. M40403 was able to positively affect in a dose dependent manner the weight gain of CII-immunized rats (FIG. 10).

Effect of M40403 on an Humoral Immunological Component of CIA

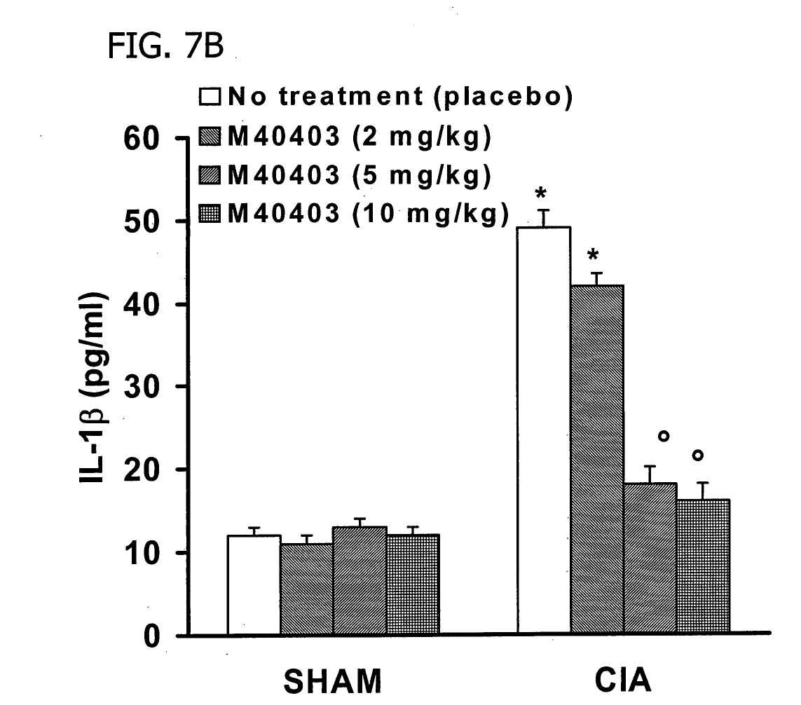

A highly significant (P<0.01) increase in serum anti-CII antibody titers was noted in CIA rats at 35 days post CII immunization (FIG. 11). M40403 had no significant effect on anti-CII antibody formation. Negligible anti-CII antibody titers were found in the serum of control rats (FIG. 11).

Discussion

The present invention demonstrates that M40403 is highly protective in a rat model of collagen-induced arthritis. The protective effects of M40403 were not limited to an overall anti-inflammatory effect but included significant protection of cartilage/bone compared to untreated collagen-immunized animals, as well as inhibition of key pro-inflammatory cytokines known to be involved in the human disease.

Through both histological and radiographical evaluations, it was found that M40403 was significantly protective on the cartilage and bone in tibiotarsal joints of rats immunized with CII.

Taken together, these examples indicate that O₂^{•-} generated at the osteoclast-bone interface plays a role in bone matrix degradation.

Besides their key role on cartilage and bone, superoxide anions exhibit several pro-inflammatory properties. Importantly, superoxide releases (via mechanisms not yet defined) cytokines such as tumor necrosis factor α and interleukin-1b (TNF-α and IL-1b respectively). These in turn have been implicated in the pathogenesis of RA based on the observations that anti-IL1b and anti-TNFa therapies suppress the development of CIA. These cytokines are not only pro-inflammatory but also mediate cartilage and -bone destruction. A role for TNF-α in the human disease has recently been shown. Thus, two anti-TNF-α therapies, Infliximab (Remicade, Centocor, Malvern, Pa.) and Etanercept (Enbrel, Immunex, Seattle, Wash.) have shown beneficial effects in patients with RA. Thus, of TNF-α is both anti-inflammatory and disease modifying. Administration of recombinant human IL-1 receptor antagonist (IL-1Ra) in patients with active RA was also found to be somewhat beneficial.

In the present invention, we show that the increase in TNF-α and IL-1b in the plasma of untreated rats with CIA-induced arthritis were reduced almost to basal levels in rats treated with M40403. Without being bound to a particular theory, it is proposed that part of the beneficial anti-inflammatory and cartilage/bone protective effects of M40403 may be mediated through ROS reduction and the prevention of or inhibition of TNF-α and IL-b. This, in turn, would lead to reduced free-radical production and subsequent damage. Interestingly, IL-1b mediated cartilage matrix degradation is blocked by SOD, indicating a potential role of O₂^{•-} in the IL-1b driven cartilage damage.

A predominant mechanism by which superoxide mediates its effects is through the diffusion-limited reaction with NO to generate peroxynitrite, a potent cytotoxic and pro-inflammatory molecule. Levels of nitrotyrosine, a marker of peroxynitrite formation, are elevated in synovial fluids in patients with RA consistent with a possible role for peroxynitrite, ONOO⁻, in human disease. Superoxide and peroxynitrite cause DNA single-strand damage, the obligatory trigger for PARP (a nuclear enzyme involved in DNA repair). Hydroxyl radical and ONOO⁻ or peroxynitrous acid (ONOOH) also induce cellular injury partially related to the development of DNA single strand breakage. Excessive activation of PARP can rapidly deplete cellular energy stores, leading to cell death. Therefore, PARP activation is an important indicator that O₂^{•-} and ONOO⁻ are mediating cytotoxic/tissue damaging effects in acute and chronic inflammatory diseases. The role of PARP in arthritis has been shown through pharmacological and genetic manipulations. Thus, inhibitors of PARP activation such as 5-iodo-6-amino-1,2-benzopyrone were protective in a mouse model of CIA and PARP knockout mice are resistant to the development of CIA.

In the present study, significant staining for nitrotyrosine and PAR was found in the inflamed joints of untreated CII-immunized rats, and this was attenuated by M40403. These findings indicate that inhibition of peroxynitrite formation and O₂^{•-}/ONOO⁻ driven PARP activation contribute to the overall protective effects of M40403 in CIA, a result consistent with the possible roles of superoxide and peroxynitrite in arthritis.

M40403 had no effect on the increase in serum anti-CII antibody titers, suggesting that it's beneficial effects are not associated with immunosuppression.

Thus, M40403 when given at the onset of the disease significantly reduced paw swelling, clinical score and the histological/radiographical severity of the disease when injected after the onset of clinical arthritis. Amelioration of joint disease was associated with near to full inhibition of TNF-α and IL-1b as well as inhibition of peroxynitrite and PARP activation, key players in RA. Thus, removal of superoxide is anti-inflammatory and results in significant protection at the level of cartilage and bone.

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

### Carrageenan Paw Hyperalgesia Testing

Sprague-Dawley rats (175-200 g, Harlan Sprague Dawley, Indianapolis, Ind., USA) were housed and cared for under the guidelines of the Institutional Animal Care and Use Committee. They received a subplantar injection of carrageenan (0.1 mL of a 1% suspension in 0.85% saline) into the right hind paw. At three hours post-carrageenan, when hyperalgesia is normally at a maximum, the test compound was administered intravenously at dosages of from 1-6 mg/kg. Hyperalgesia is assessed at thirty minutes to three hours post-administration of test compound.

### Example 2

### Induction of Collagen-Induced Arthritis

Male Lewis rats (160-180 g; Charles River; Milan; Italy) were used for these studies. Collagen-induced arthritis was induced as described in Griffiths M. M. et al., Immunogenetic Control of Experimental Type II Collagen-induced Arthritis. 1. Susceptibility and Resistance among Inbred Strains of Rats, Arthritis Rheum. (2): 781-789 (1981) and Tawara T. et al., Effects of Recombinant Human IL-1b on Production of Prostaglandin E2, Leukotriene B4, NAG, and Superoxide by Human Synovial Cells and Chondrocytes, Inflammation (15):145-57 (1991). Bovine type II collagen (CII, Sigma) was dissolved in 0.1 M acetic acid at a concentration of 2 mg/ml by stirring overnight at 4° C. Dissolved CII was frozen at −70° C. until use. Rats were immunized with an emulsion containing 2 mg/ml of CII in Incomplete Freund's adjuvant (IFA). The emulsions were prepared by homogenizing one part CII into one part IFA (Sigma) at 4° C. On day 1, rats were injected intradermally at the base of the tail with 100 ml of the emulsion. On day 21, a second injection of CII in IFA was administered at the base of the tail.

### Example 3

### Suppression of Collagen-Induced Arthritis by M40403

Animals were randomly divided into five groups (n=16 for each group). The first group (Group 1) was injected intraperitoneally (i.p) with vehicle only (26 mM sodium bicarbonate buffer, pH 8.1-8.3) and served as a naive group. Collagen-induced arthritis was elicited in groups 2, 3, 4 and 5. In groups 3, 4 and 5 rats were treated with M40403 at 2, 5 and 10 mg/kg respectively. M40403 was given intraperitoneally every 24 h starting from day 25. Group 2 received an equivalent volume of vehicle. Rats were evaluated daily for clinical signs of arthritis using a macroscopic scoring system which is based on redness/swelling/deformity of the joint: 0=no signs of arthritis; 1=swelling and/or redness of the paw or one digit; 2=two joints involved; 3=more than two joints involved; and 4=severe arthritis of the entire paw and digits. Arthritic index score for each rat was calculated by adding the four scores of individual paws. The Mean Arthritic Score (MAS) for each rats was calculated by dividing the total number of points scored by the group by the number of animals in the group. Clinical severity was also determined by quantitating the change-in the paw volume using plethysmometry (model 7140; Ugo Basile).

### Example 4

### Assessment of Arthritis Damage

At day 35, animals were euthanized under anesthesia, and paws and knees were removed and fixed in 10% formalin for microscopic histological evaluation. The paws were then trimmed, placed in decalcifying solution for 24 h, embedded in paraffin, sectioned at 5 mm, stained with trichromic Van Gieson and studied using light microscopy (Dialux 22 Leitz). The following morphological criteria were considered by an investigator blinded for the treatment regime: score 0, no damage; score 1, sloughing of the articular space; score 2, inflammatory cell presence; score 3, bone erosion.

Histomorphometric analysis was carried out in the proximal tibia near the joint on 5 mm thick sections, using a morphometry software, a computer with a digitizing board and a Nikon Labophot microscope equipped with both visible and UV light sources and a camera lucida attachment. Parameter for histomorphometry employed in this study, derived from Parfitt and colleagues, have been approved by an ASBMR committee. See Parfitt A. M. et al., Bone Histomorphometry: Standardization of Nomenclature, Symbols and Units, J. Bone. Miner. Res. (2):596-610 (1987). To measure bone formation, osteoblast surface was quantified relative to bone surface (Ob/Bs). To measure bone resorption, eroded surface, osteoclast surface, were quantified relative to bone surface (ES/Bs, Oc.S/Bs).

### Example 5

### Radiography

The rats were anaesthetized with sodium pentobarbital (45 mg/kg, i.p.). Rats were placed on a radiographic box at a distance of 90 cm from the x-ray source. Radiographic analysis (Philips X12 Germany) of normal and arthritic rat hind paws was performed with a 40 kW exposure for 0.01 sec. An investigator blinded to the treatment regime scored the radiographs. The following radiographic criteria from both hind limbs were considered: score 0, no bone damage; score 1, tissue swelling and edema; score 2, joint erosion; 3, bone erosion.

### Examples 6-166

The following compounds were made for use as superoxide dismutase catalysts or as ligands for combination with transition metal ions for use as superoxide dismutase catalysts within the scope of the invention. The catalytic rate constant k_{cat} is given for each compound. For k_{cat} values marked with an asterisk, the k_{cat} was measured at a pH of 8.1. For all other compounds the k_{cat} was measured at pH 7.4. Compounds marked NT were made but not tested. The ligands of Examples 11, 101, 123-135 and 138-148 were not expected to have activity without the metal ion and most were not tested. However, as can be seen by comparison of Examples 148 and 149, insertion of the metal ion into the ligand forms a complex with good superoxide dismutase activity.

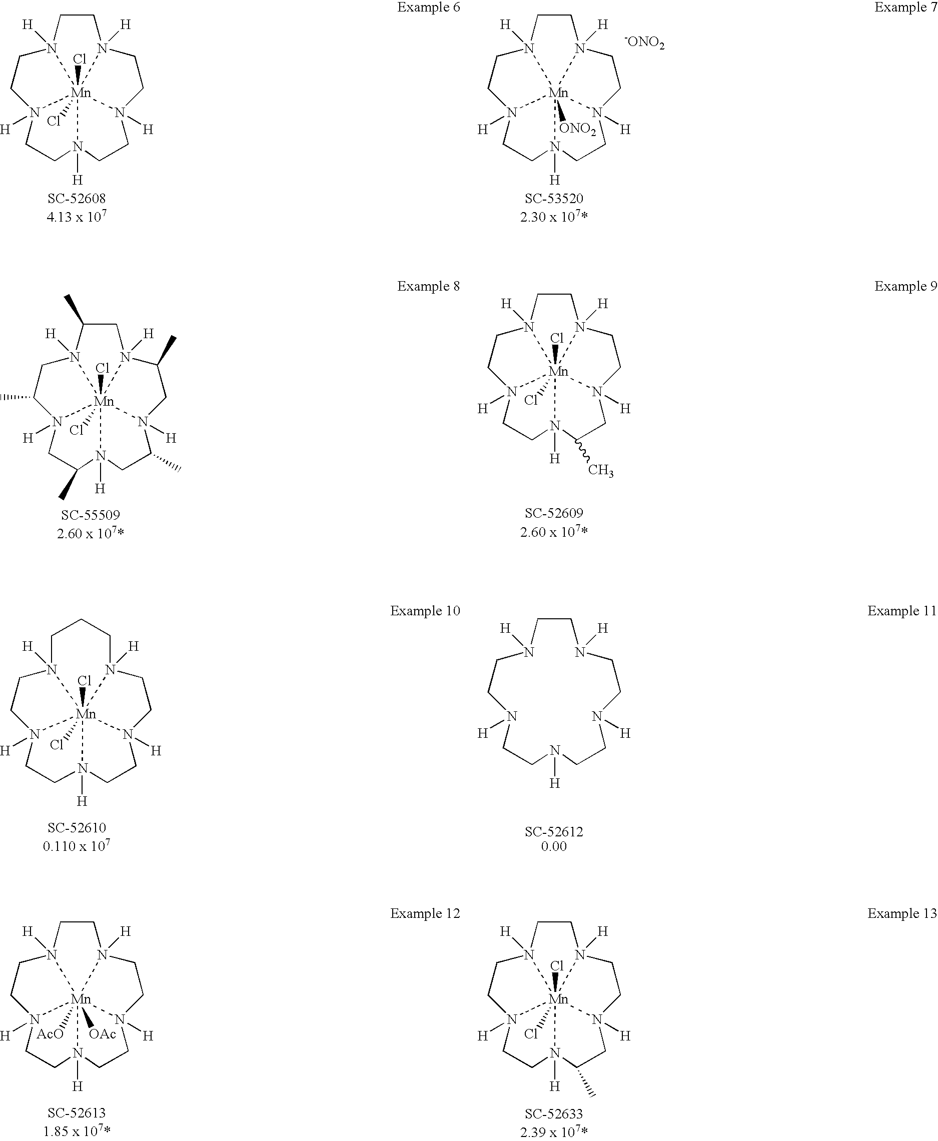

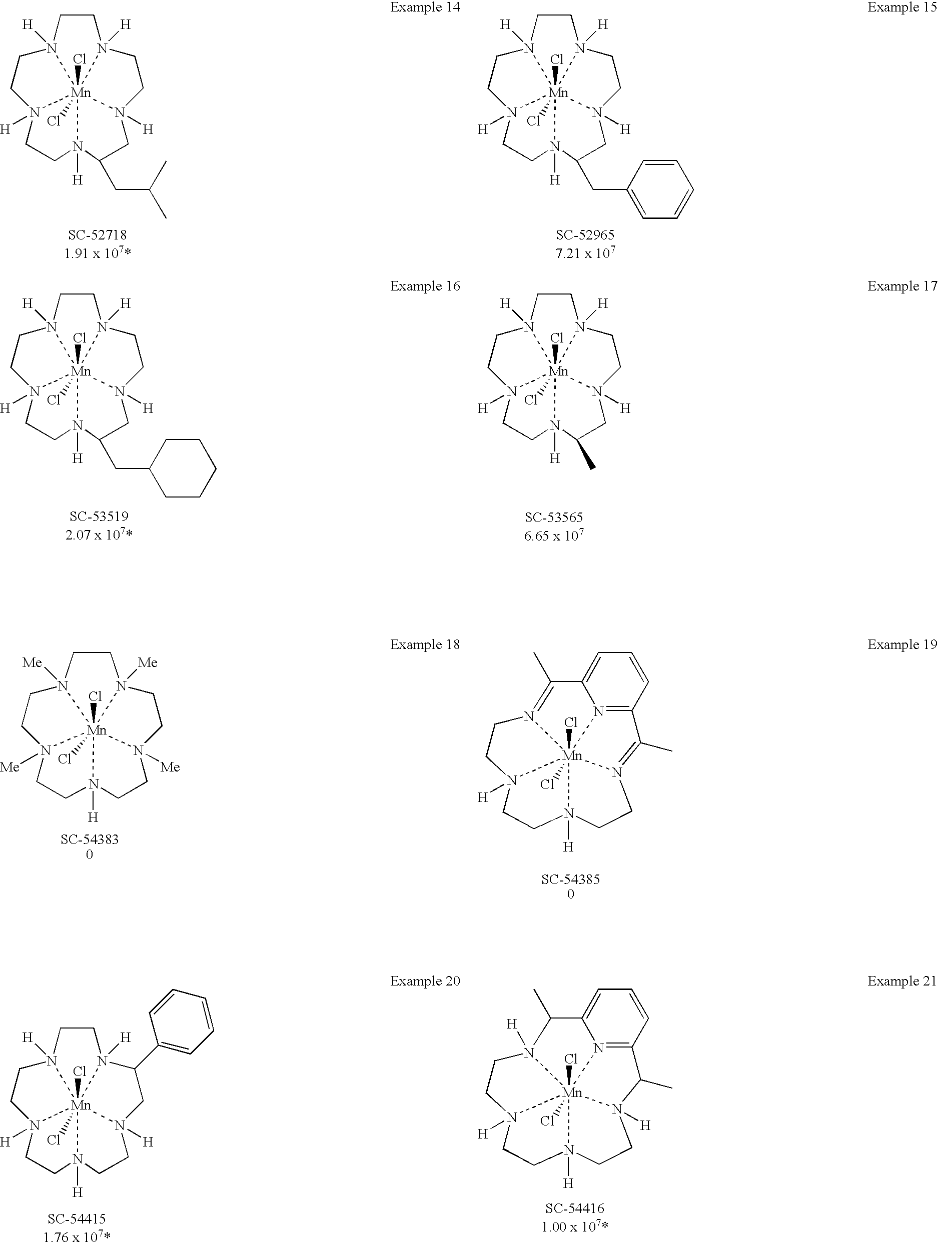

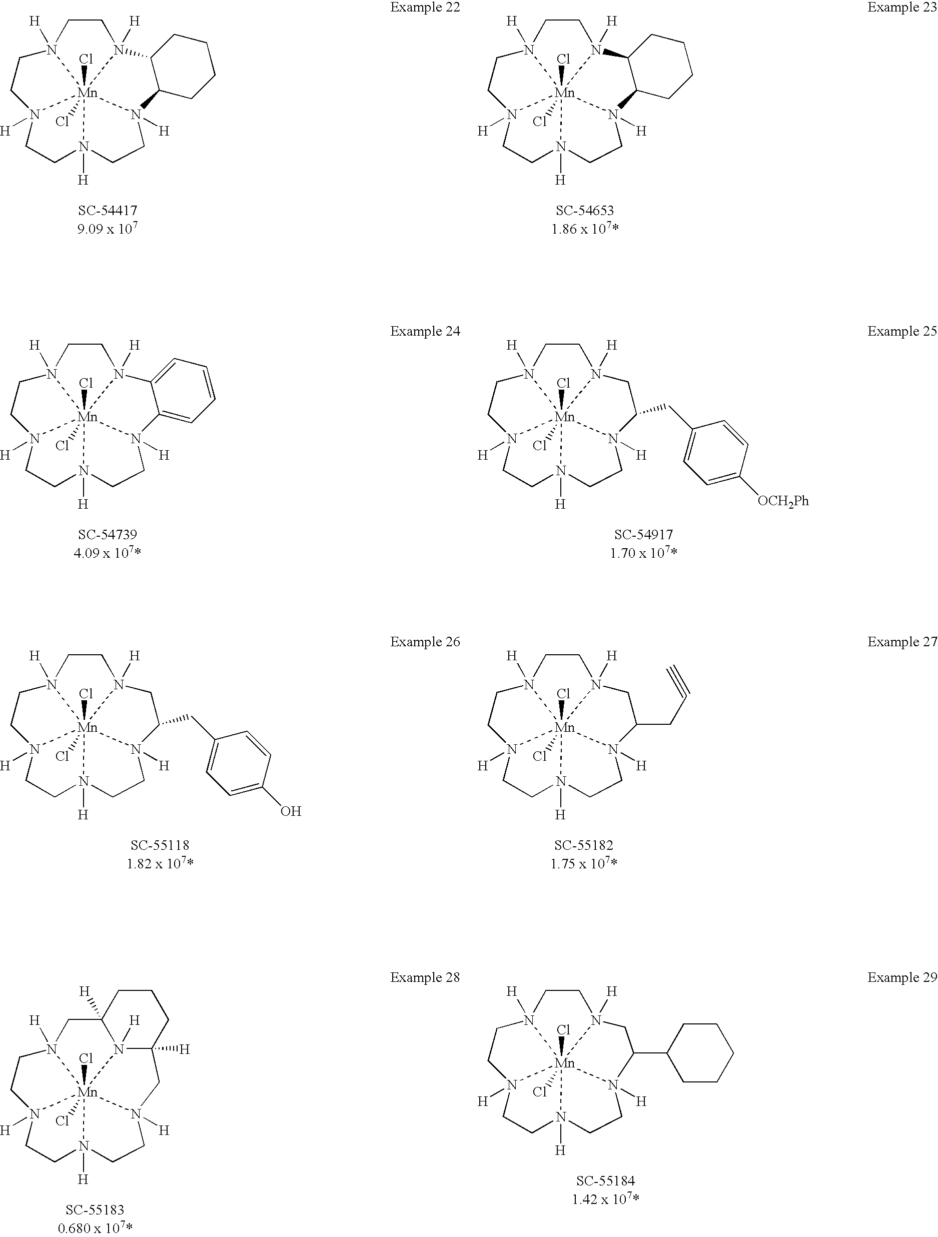

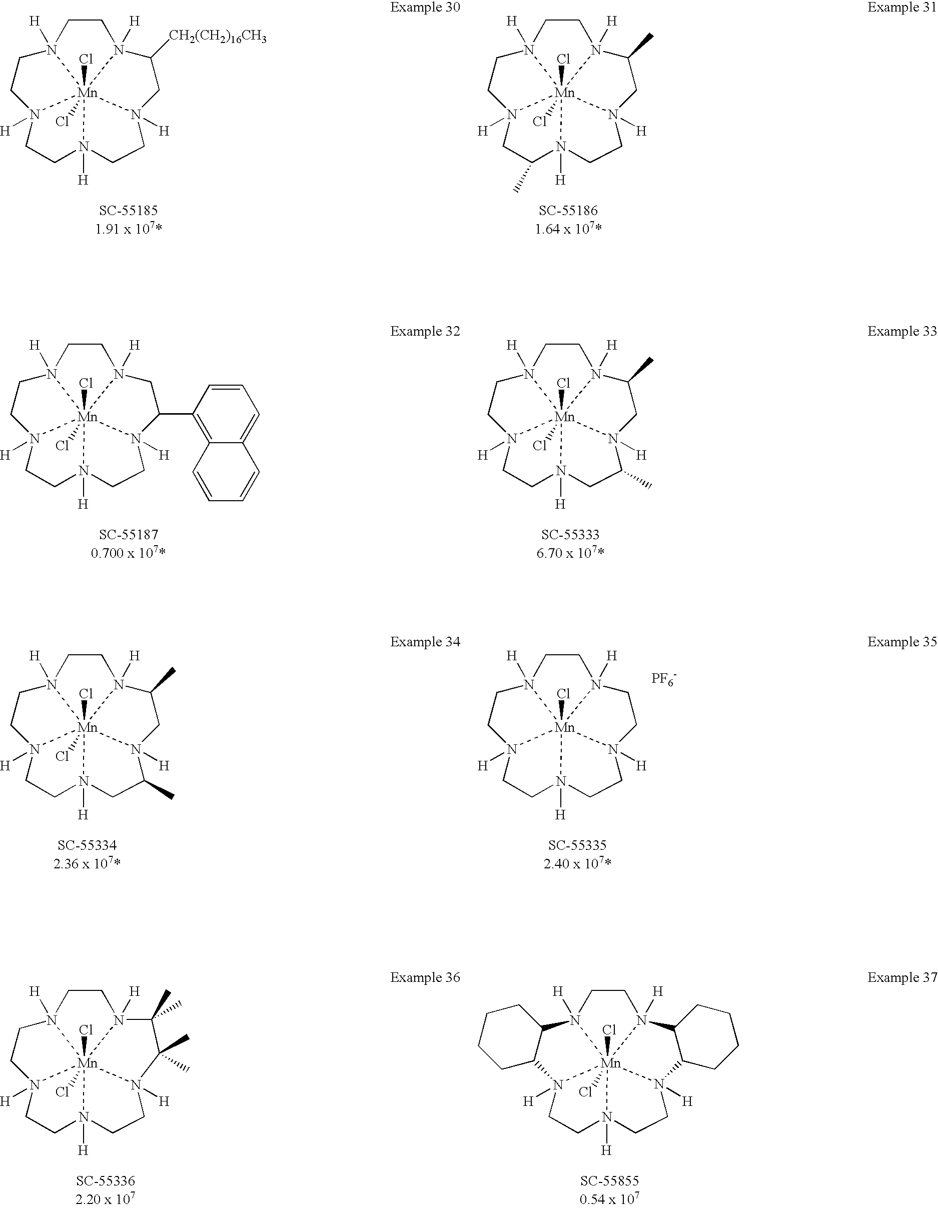

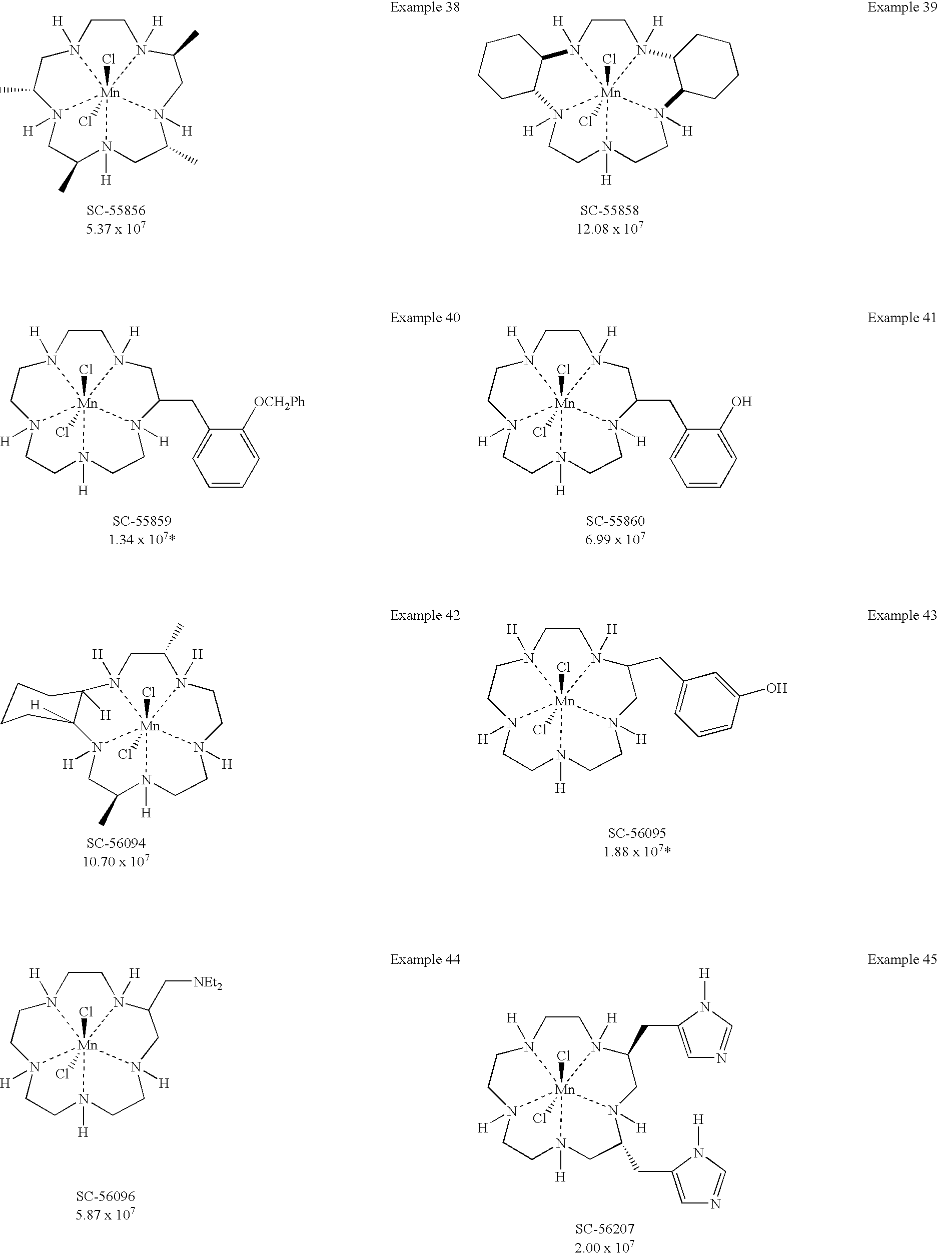

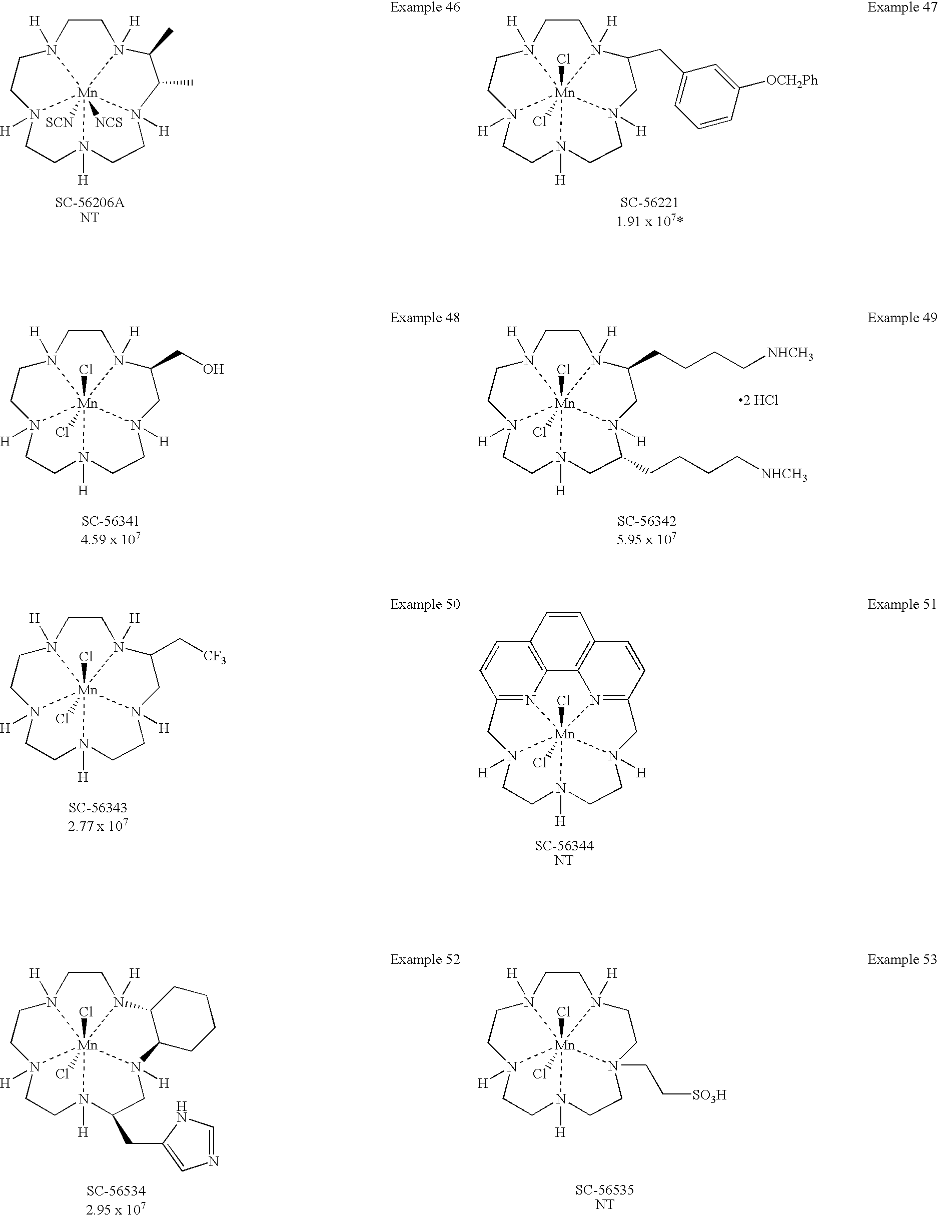

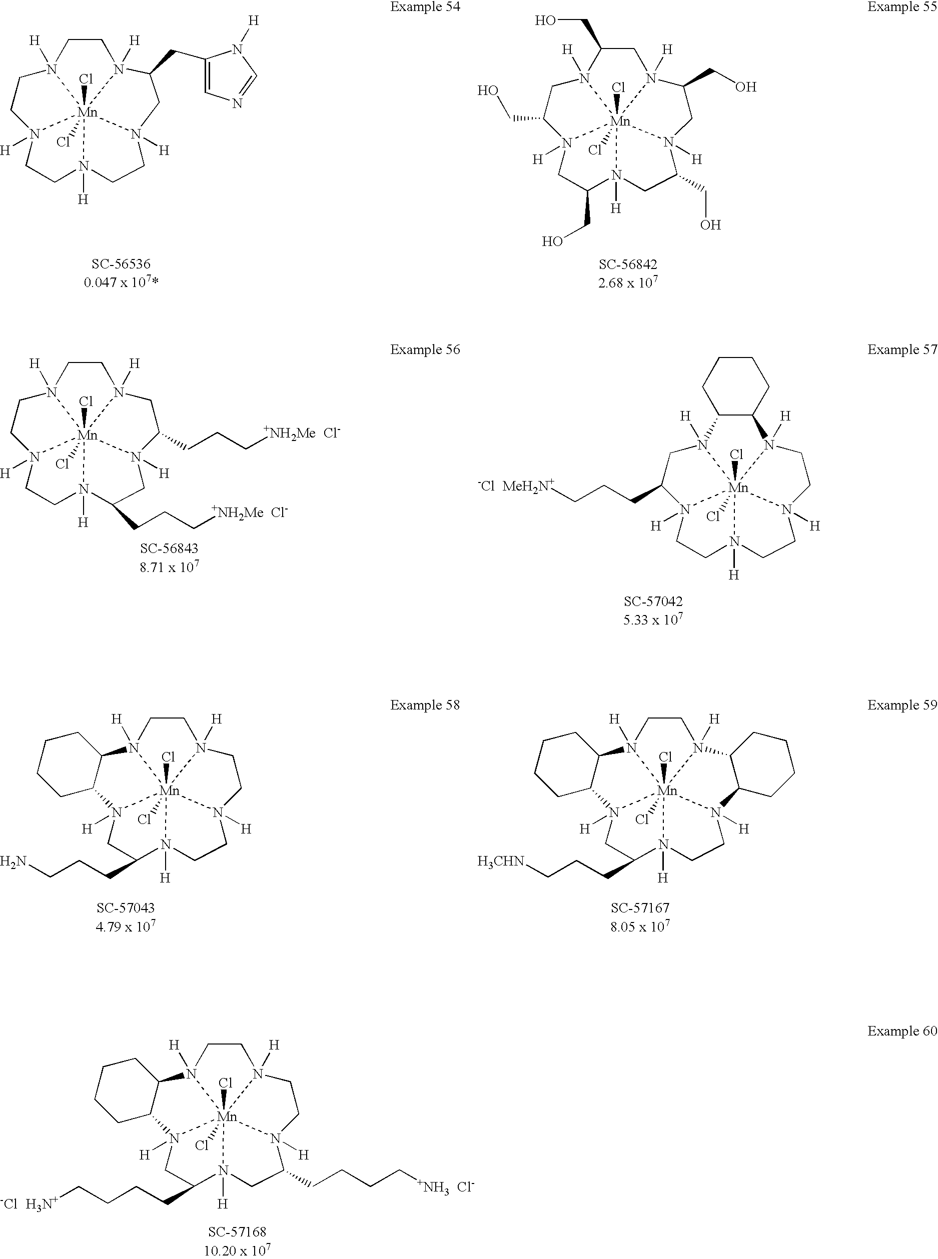

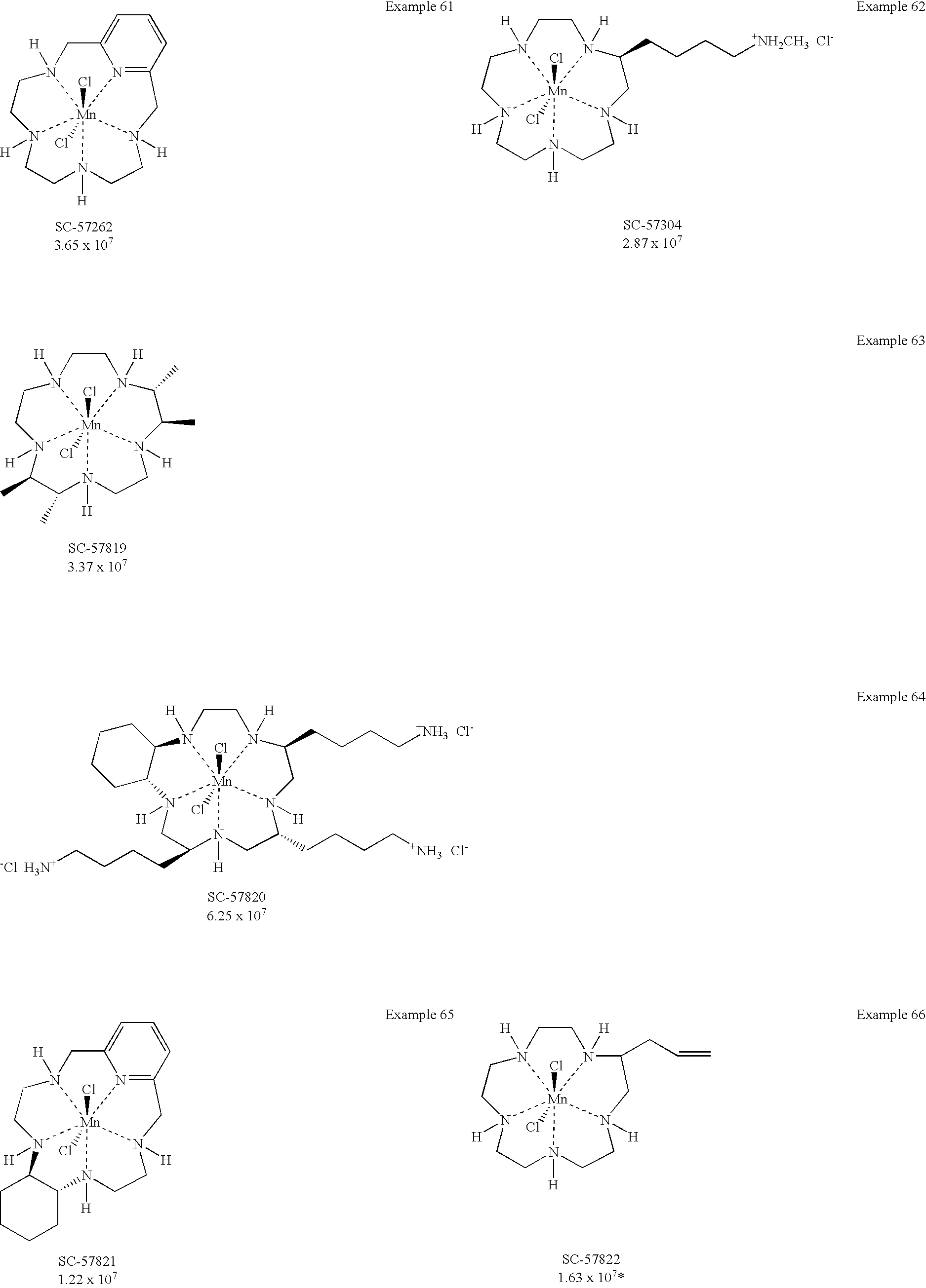

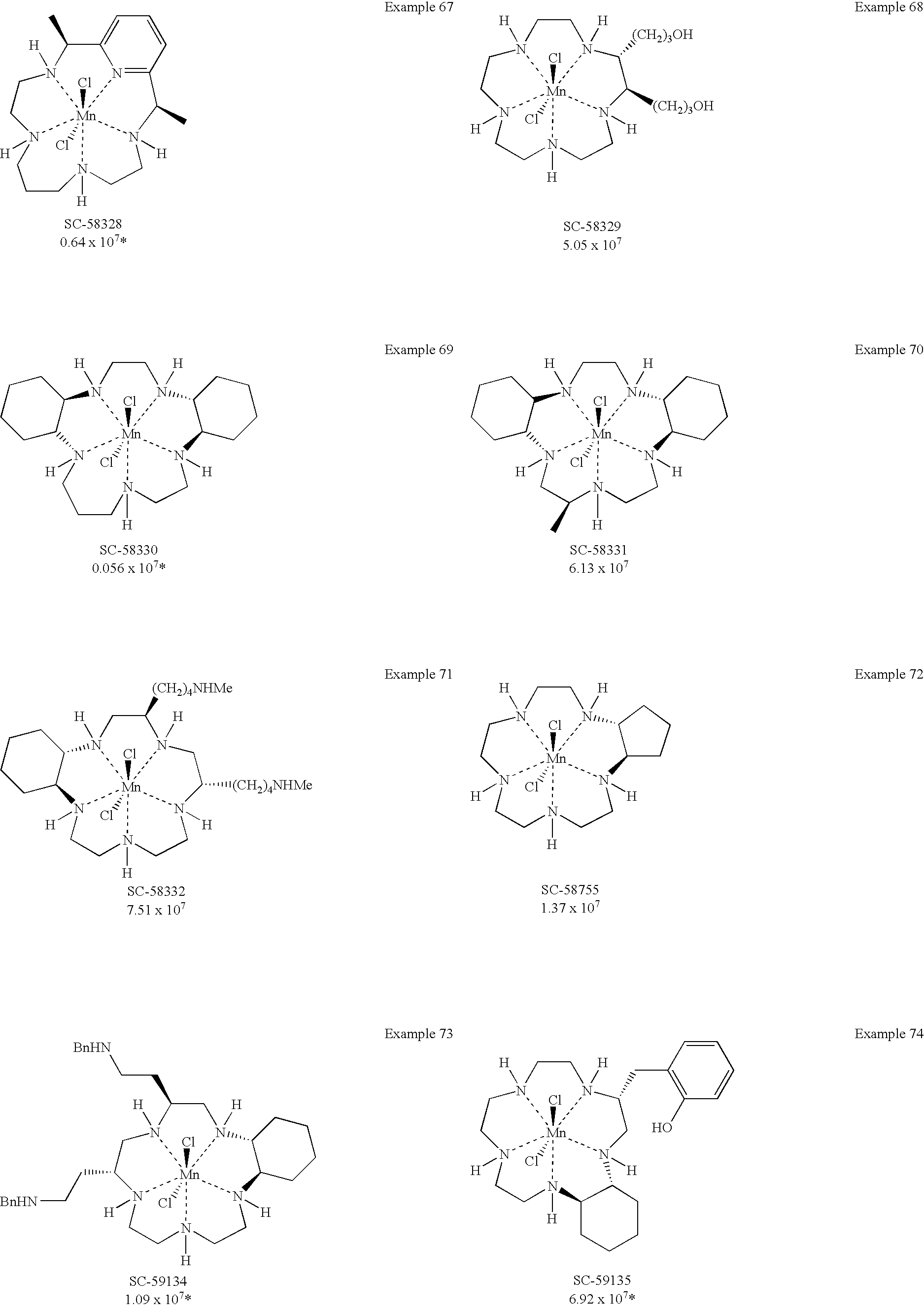

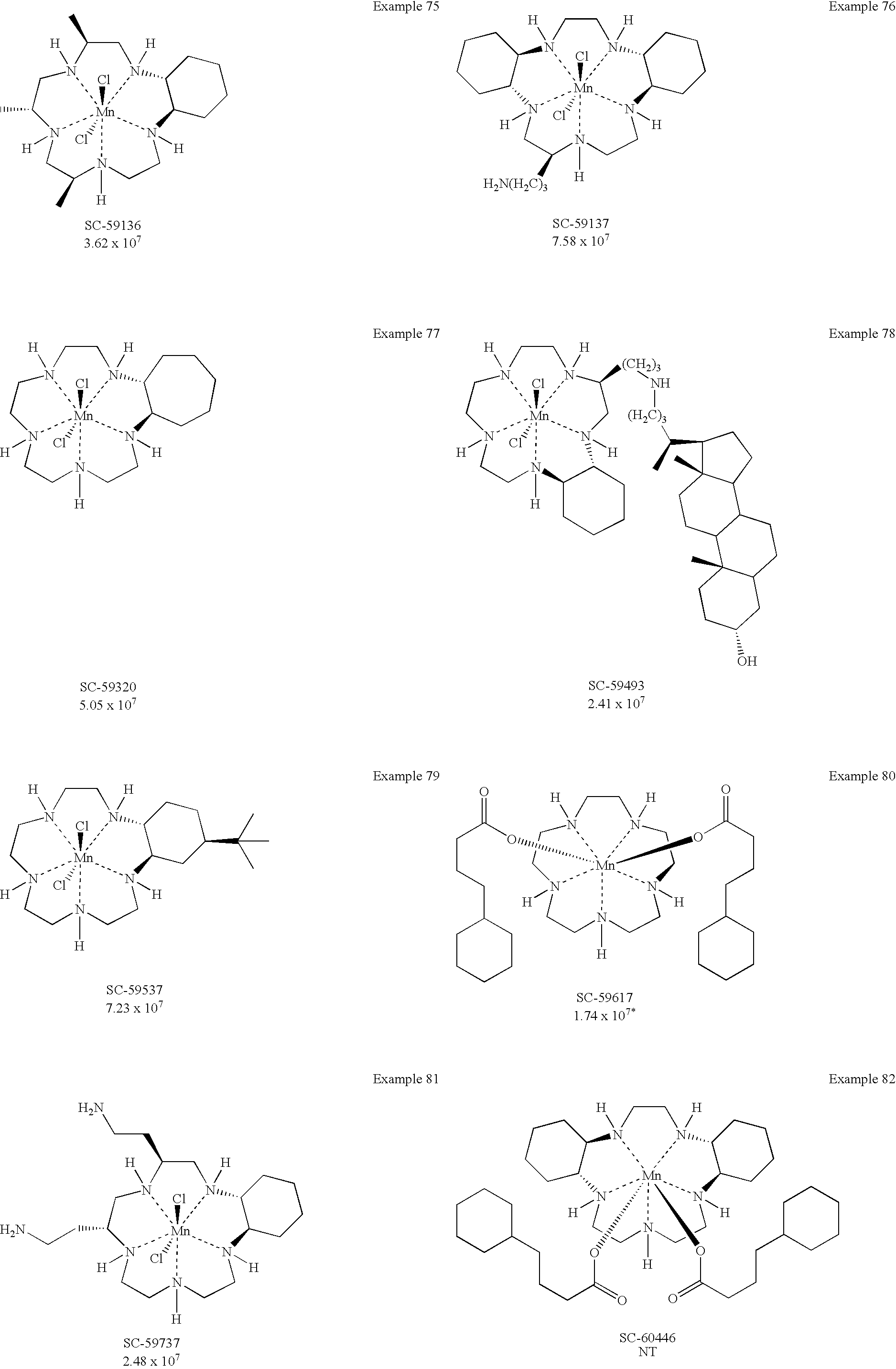

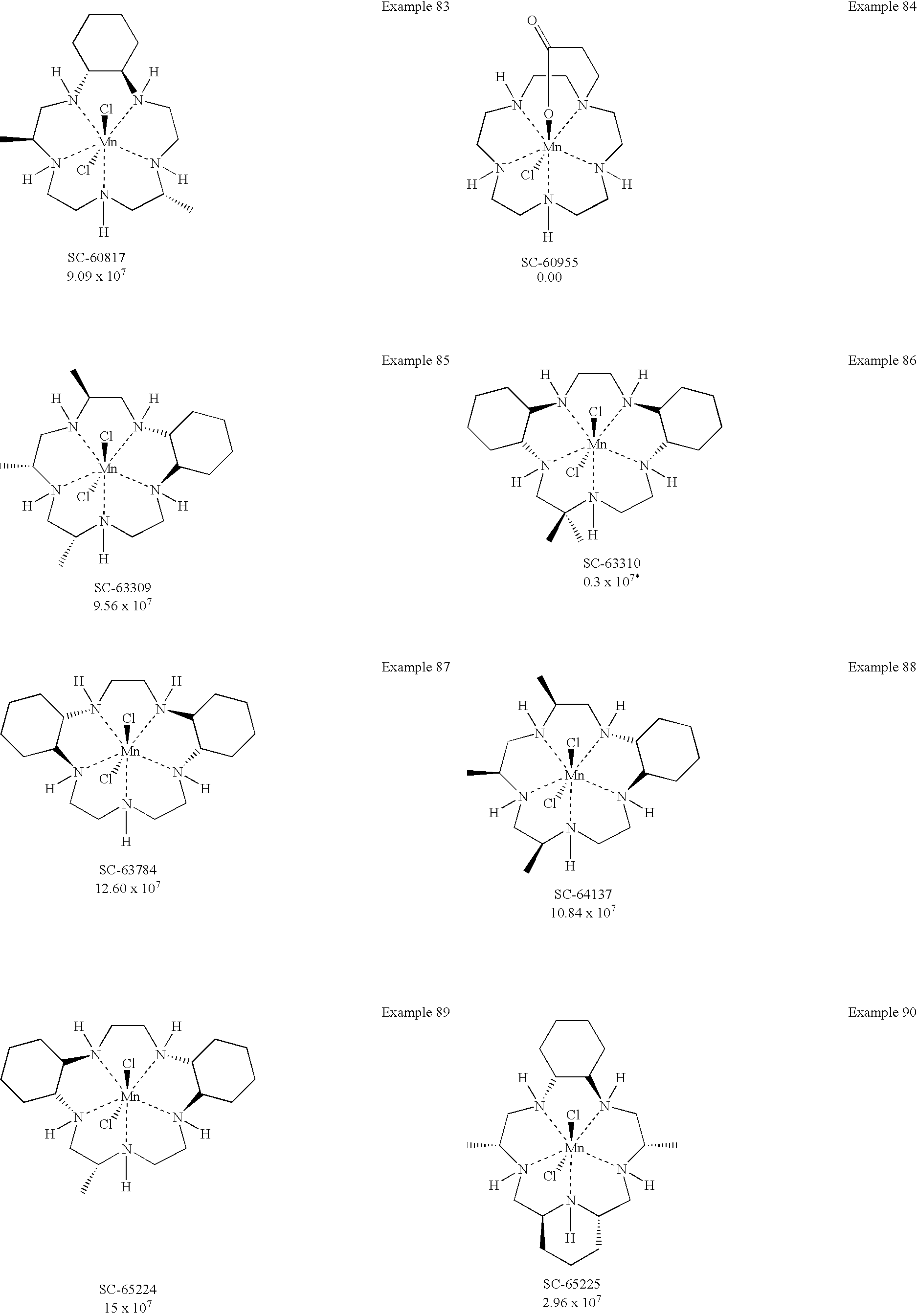

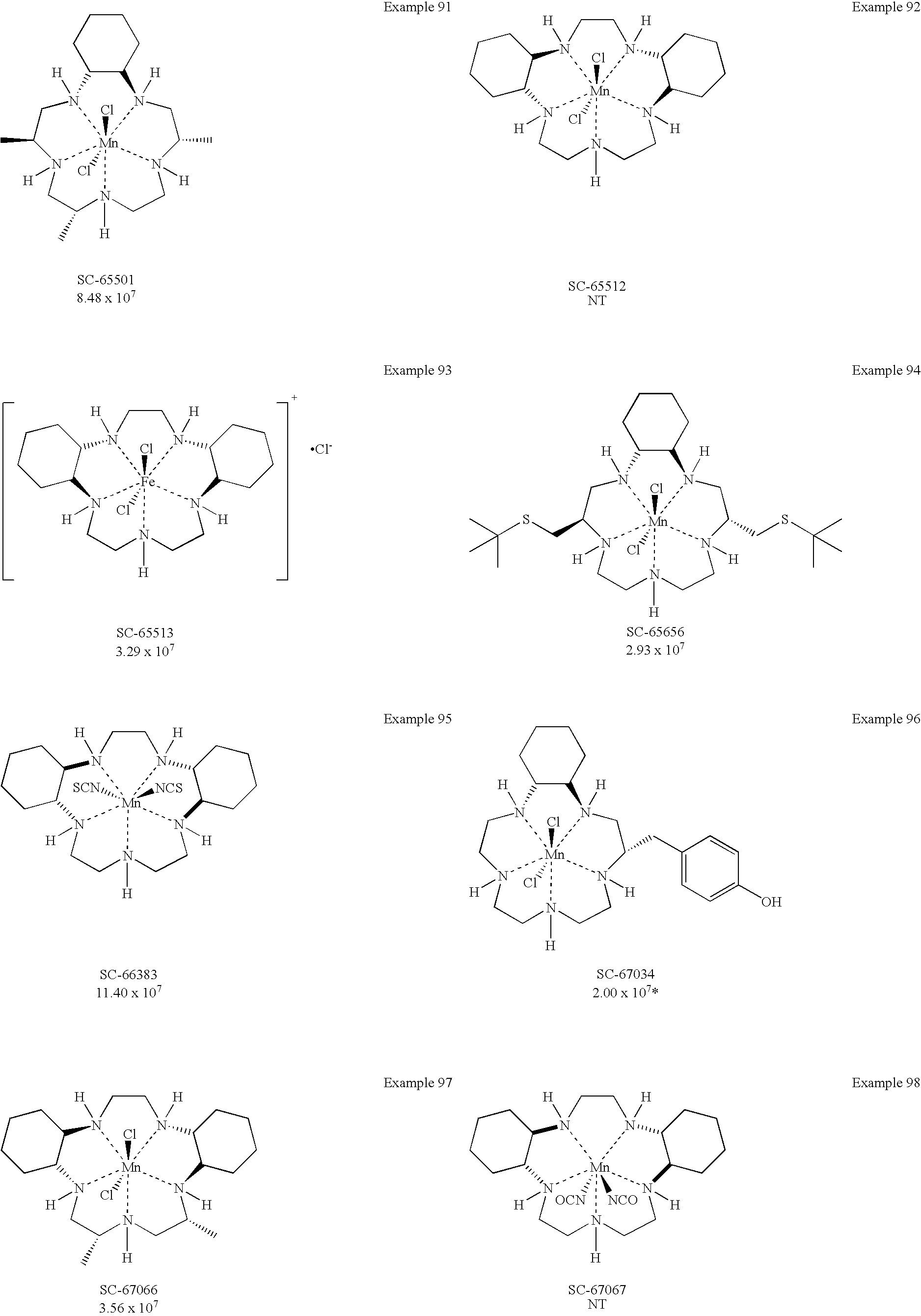

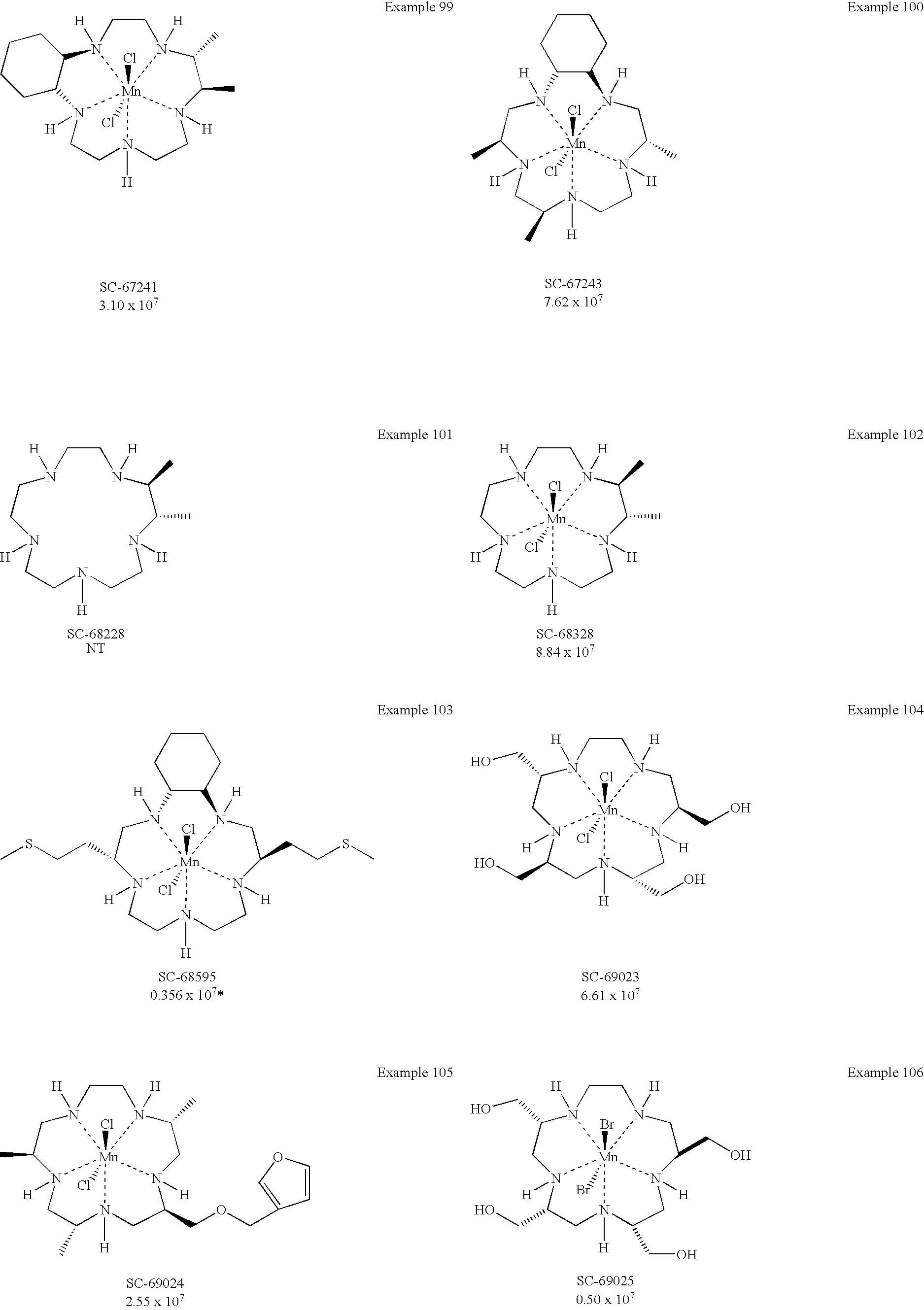

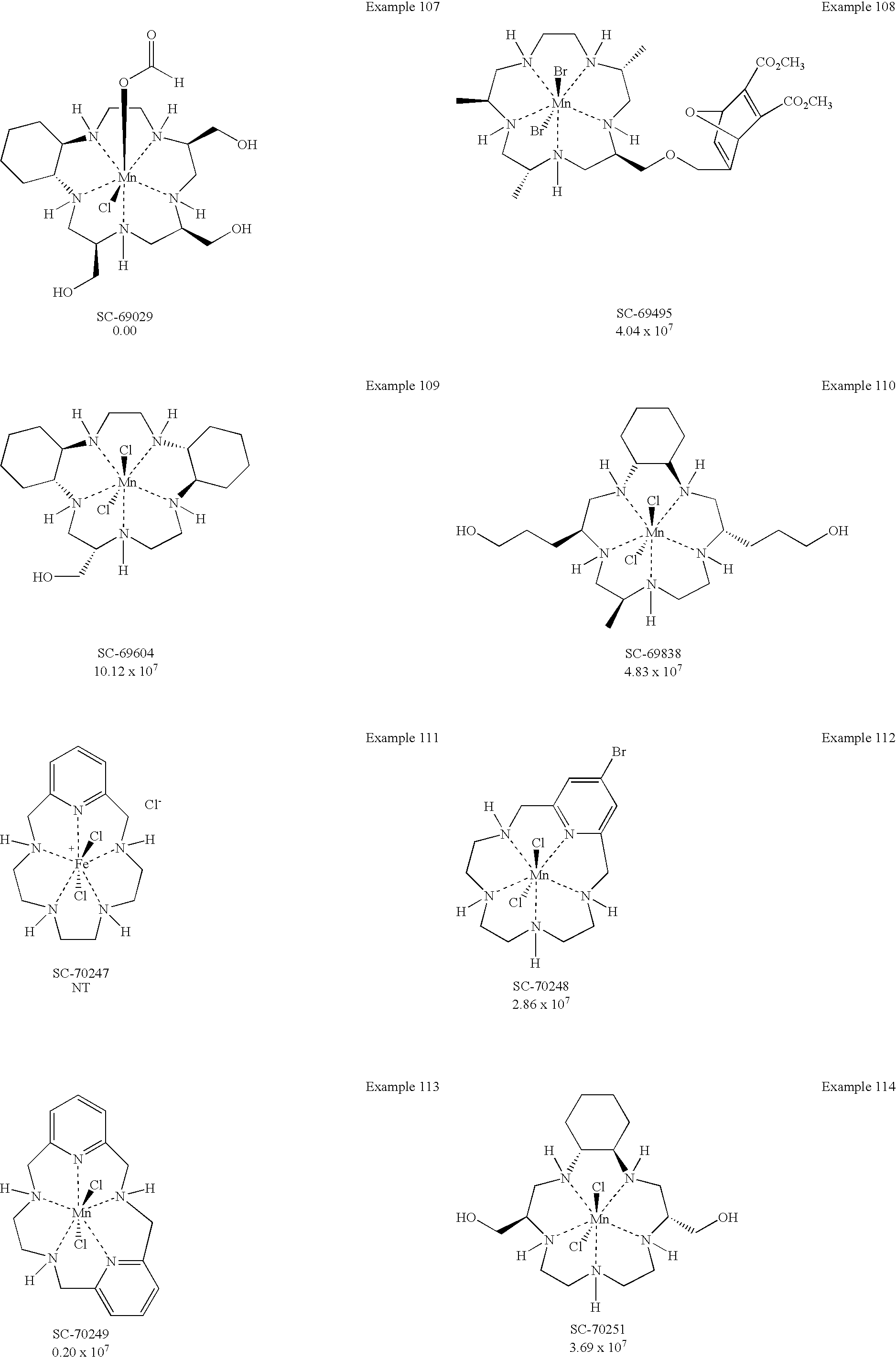

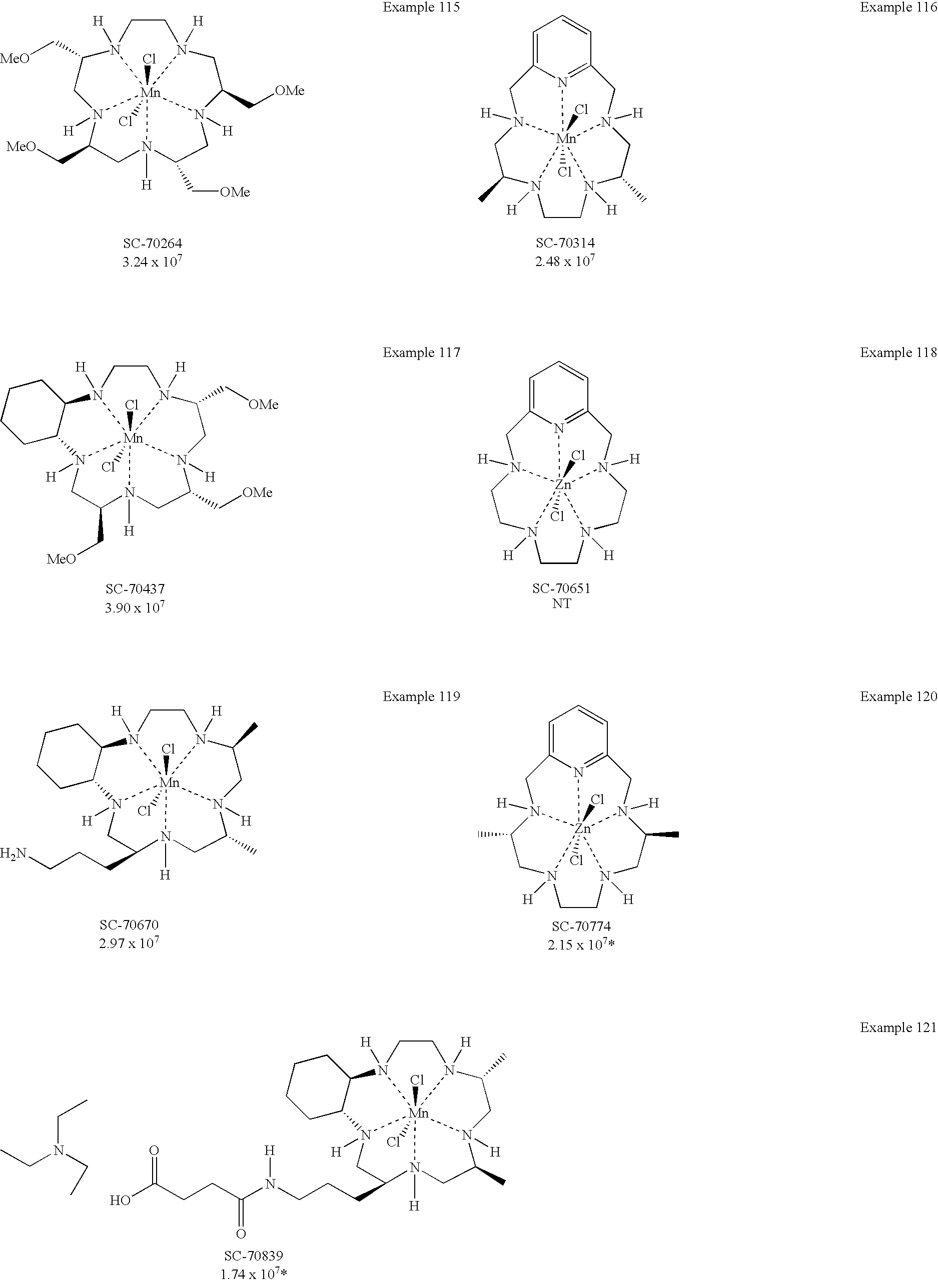

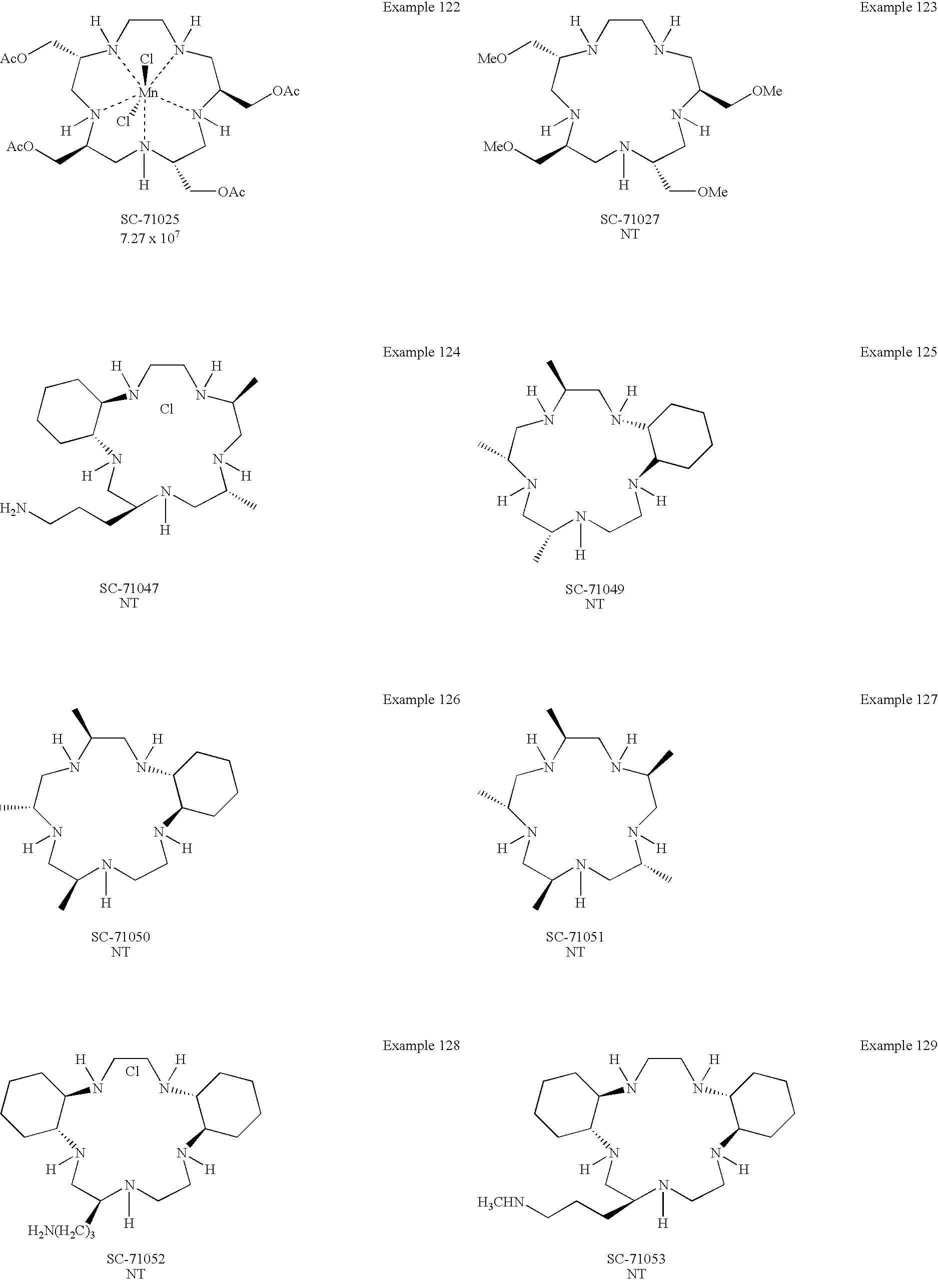

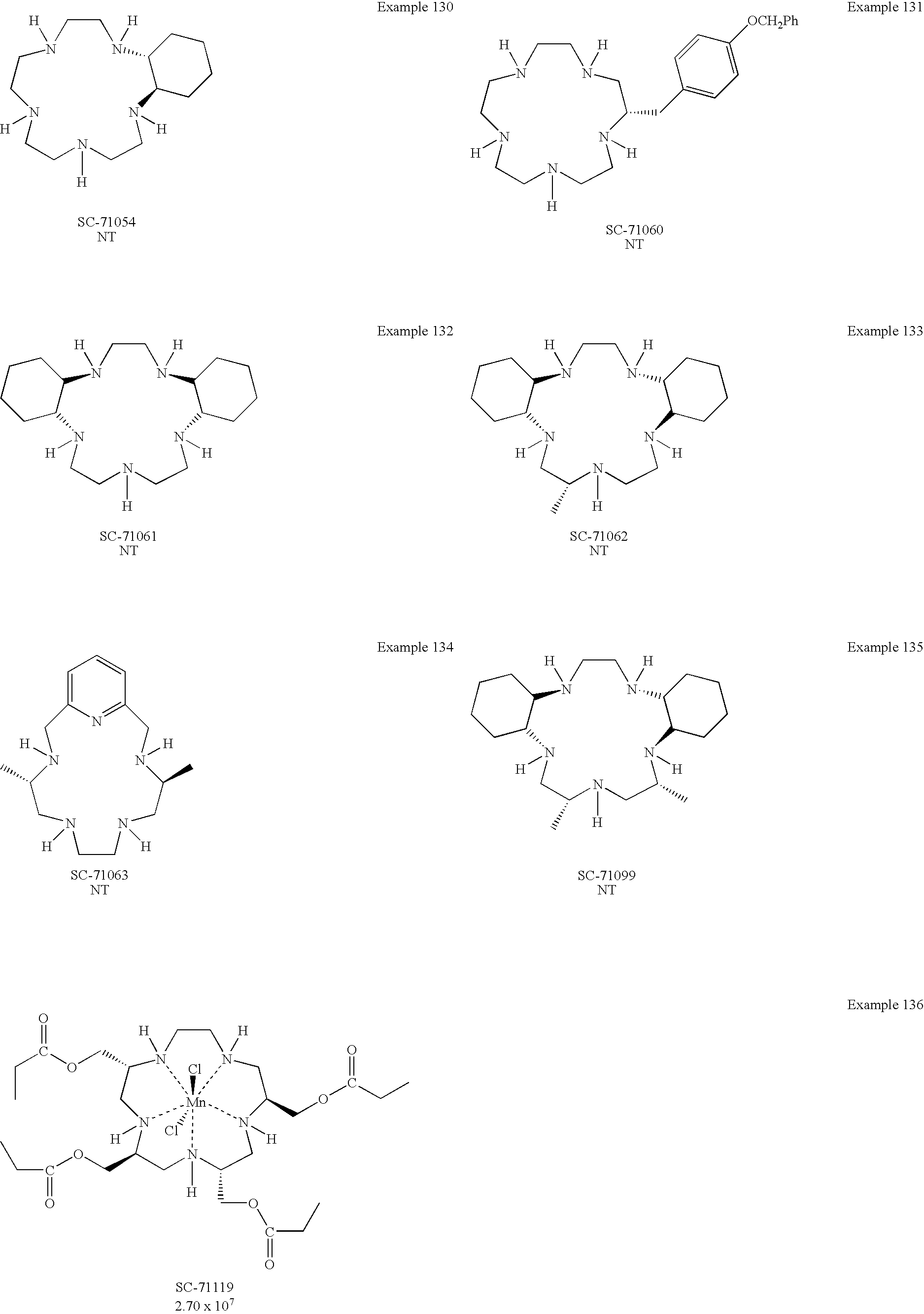

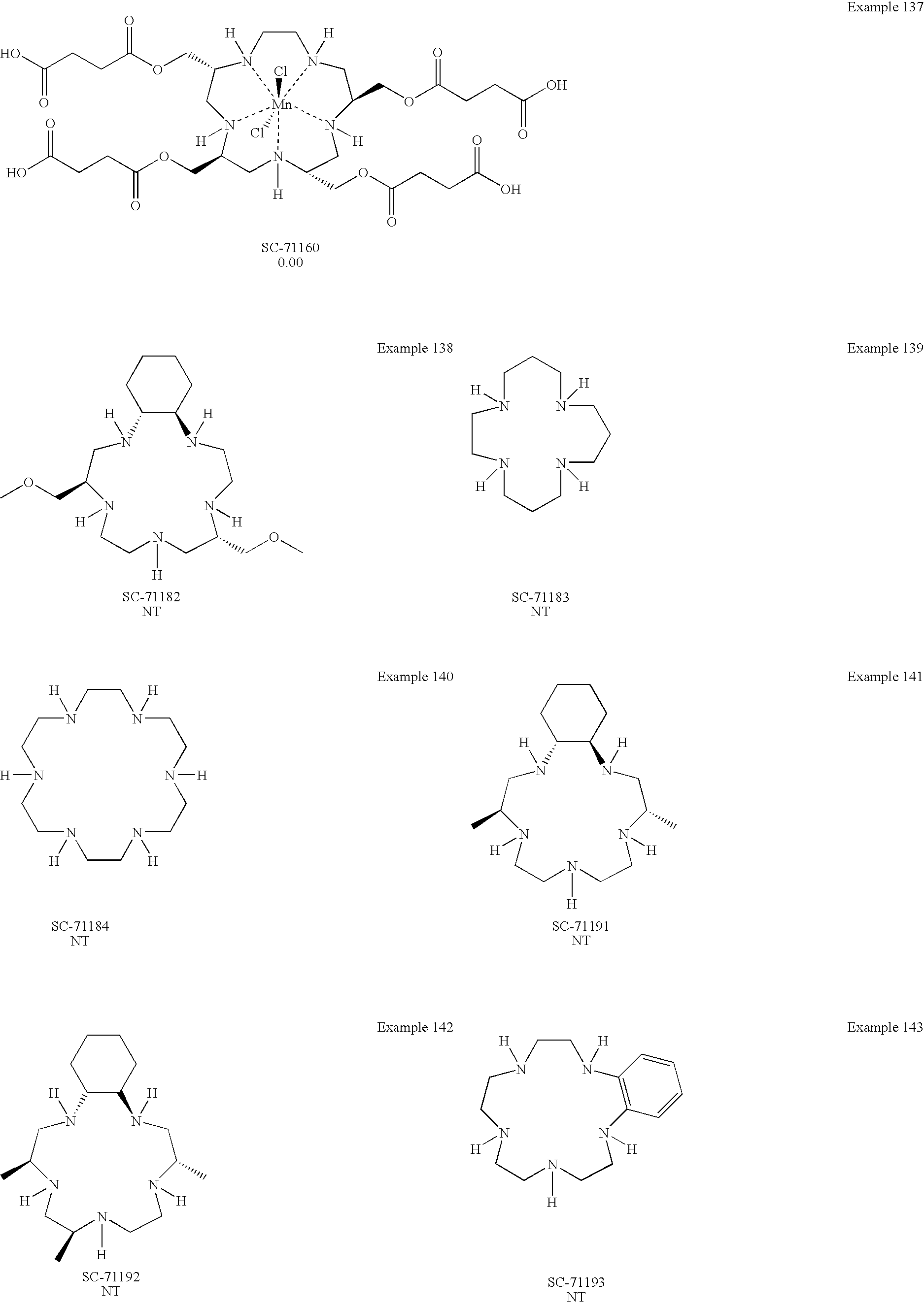

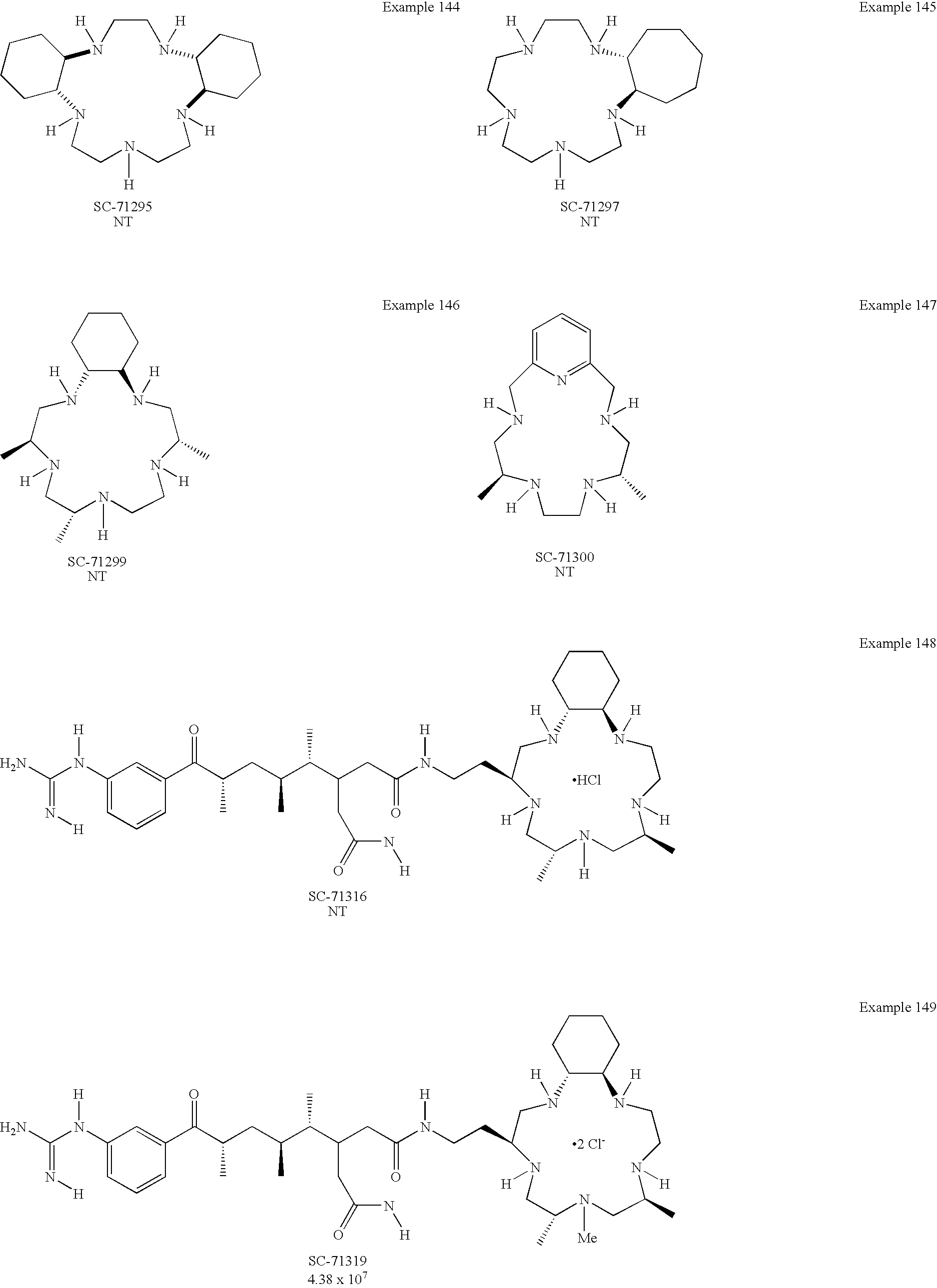

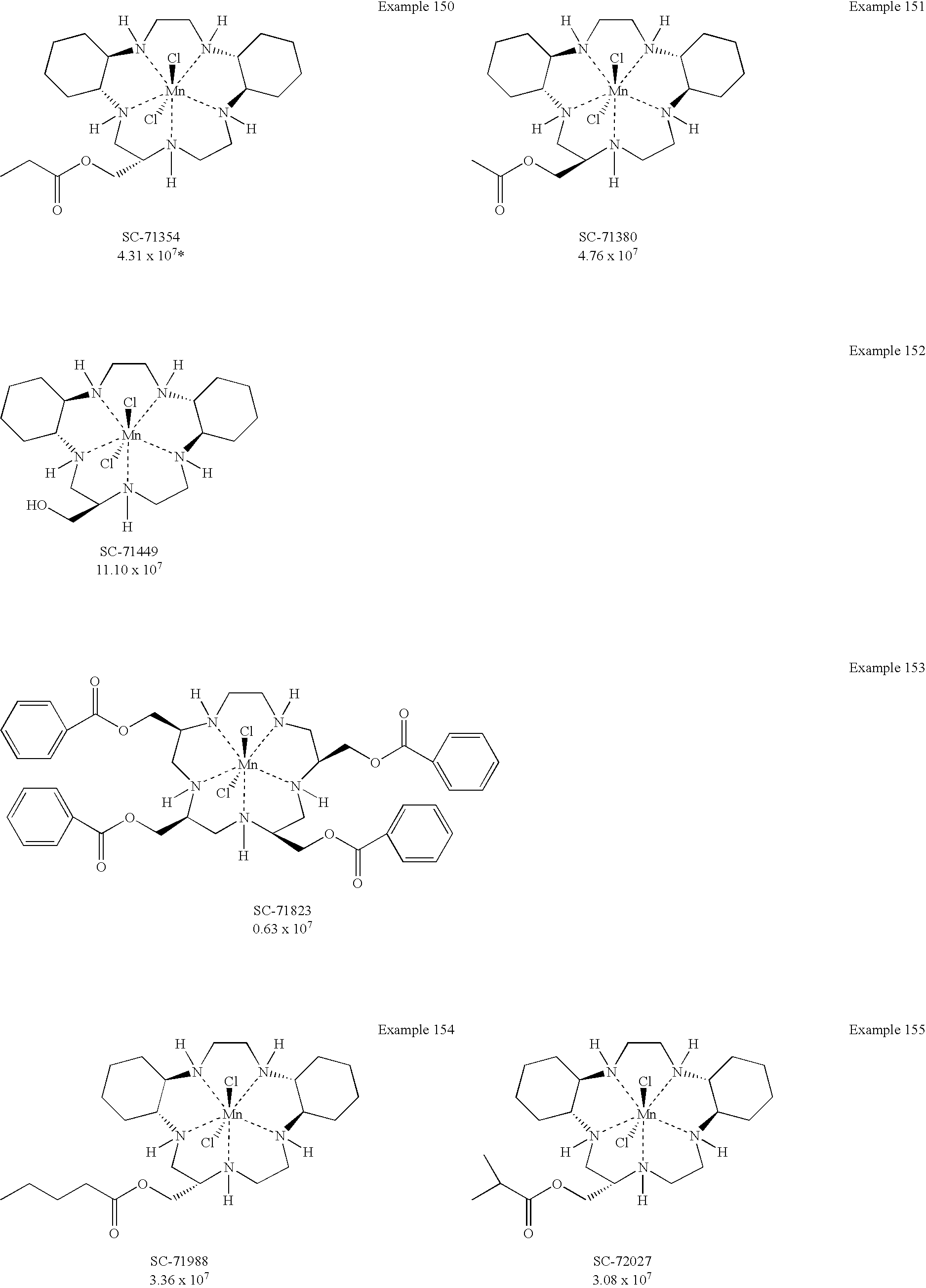

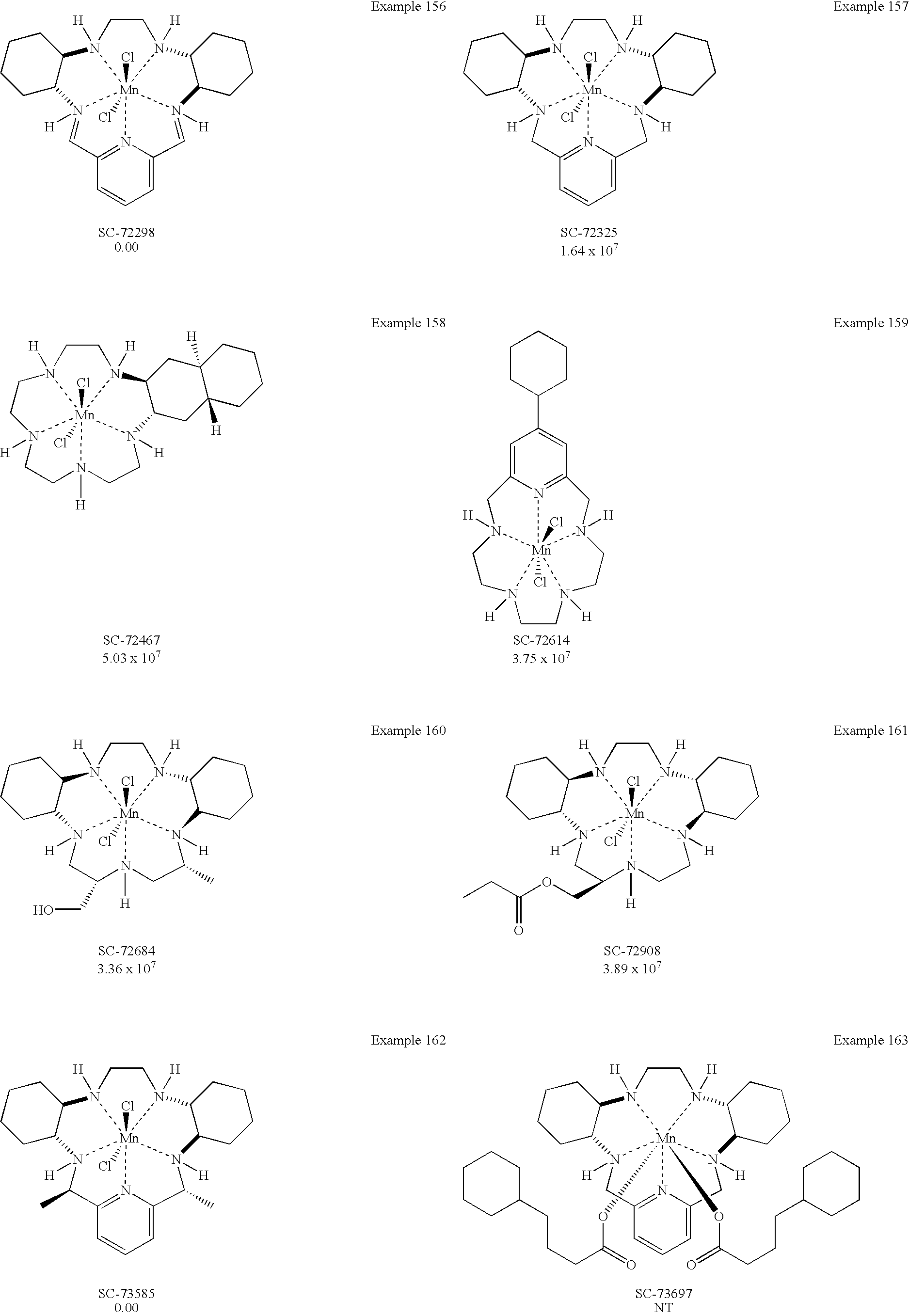

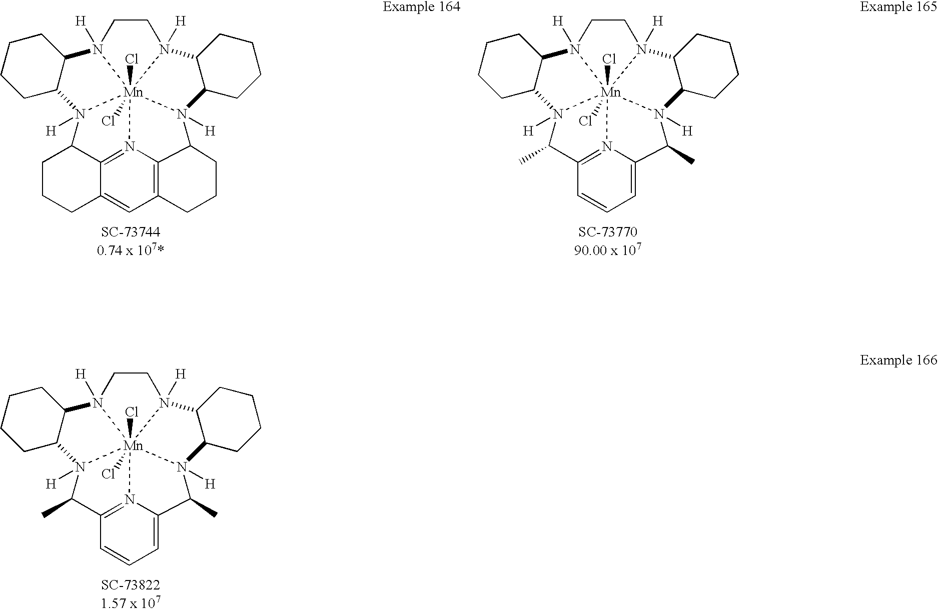

### Example 167

### Immunohistochemical Localization of Nitrotyrosine and PARP

Tyrosine nitration, an index of the nitrosilation of proteins by peroxynitrite and/or oxygen-derived free radicals, was determined by immunohistochemistry as previously described in Cuzzocrea S. et al., Beneficial Effects of Tempol, a Membrane-permeable Radical Scavenger, in a Rodent Model of Collagen-induced Arthritis, Arthritis Rheum. (43):320-8 (2000). At day 35, the joints were trimmed, placed in decalcifying solution for 24 h, and 8 μm 20 sections were prepared from paraffin embedded tissues. After deparaffinization, endogenous peroxidase was quenched with 0.3% H₂O₂ in 60% methanol for 30 min. The sections were permeabilized with 0.1% Triton X-100 in PBS for 20 min. Non-specific adsorption was minimized by incubating the section in 2% normal goat serum in phosphate buffered saline for 20 min. Endogenous biotin or avidin binding sites were blocked by sequential incubation-for 15 min with avidin and biotin. The sections were then incubated overnight with primary anti-nitrotyrosine antibody (1:1000) or anti-poly (ADP Ribose) (PAR) antibody (1:500) or with control solutions. Controls included buffer alone or non-specific purified rabbit IgG. Specific labeling was detected with a biotin-conjugated anti-rabbit IgG (for nitrotyrosine) or with a biotin-conjugated anti-rabbit IgG (for PARP) and avidin-biotin peroxidase complex. In order to confirm that the immunoreaction for the nitrotyrosine was specific some sections were also incubated with the primary antibody (anti-nitro tyrosine) in the presence of excess nitrotyrosine (10 mM) to verify the binding specificity. To verify the binding specificity for PAR, some sections were also incubated with only the primary antibody (no secondary) or with only the secondary antibody (no primary). In these situations, no positive staining was found in the sections indicating that the immunoreaction was positive in all the experiments carried out. All the experiments were carried out by an investigator blinded to the treatment regime.

### Example 168

### Serum Anti-CII Antibody Determination

The serum antibodies to CII were quantitated by ELISA using biotin-labeled goat anti-rat IgG (Southern Biotechnology Associates, Inc., Birmingham, Ala.) according to the method of Watson et al., Human HLA-DRb Gene Hypervariable Region Homology in the Biobreeding BB Rat: Selection of the Diabetic-resistant Subline Response to Human Type II Collagen, J. Exp. Med. (172):1331-1339 (1990). Serum was prepared from the blood of control and treated rats days post-CII immunization.

### Example 169

### Measurement of Cytokines

TNFa and IL-1b levels were evaluated in plasma at 35 days after the induction of arthritis. The assays were carried out by ELISA using a calorimetric, commercial kits (Calbiochem-Novabiochem Corporation, USA). Each ELISA has a lower detection limit of 5 pg/ml.

### Example 170

### Materials

Perchloric acid was obtained from Aldrich (Milan, Italy). Primary anti-nitrotyrosine antibody was from Upstate Biotech (DBA, Milan, Italy). M40403 was synthesized in house as described in Salvemini D. et al., Synzymes: Potent Non-peptidic Agents Against Superoxide-driven Tissue Injury, Science (286):304-6 (1999). All other reagents and compounds used were obtained from Sigma Chemical Company (Sigma, Milan, Italy).

### Example 171

### Data Analysis

All values in the figures and text are expressed as mean standard error (s.e.m.) of the mean of n observations. For the in vivo studies, n represents the number of animals studied. In the experiments involving histology or immunohistochemistry, the photos shown are representative of at least three experiments performed on different experimental days. Data sets were examined by one- and two-way analysis of variance, and individual group means were then compared with Student's unpaired t test. For the arthritis studies, Mann-Whitney U test (two-tailed, independent) was used to compare medians of the arthritic indices. Values for the in vitro studies are presented as incidences (%), or medians. A p value less than 0.05 was considered significant.

In view of the above, it will be seen that the several objectives of the invention are achieved and other advantageous results attained.

### Example 172

SOD catalyst compounds were evaluated in the carrageenan hyperalgesia model described above. Results were as follows:

| Compound | Result |
|---|---|
| SC-71354 | No effect at tested dosages by intravenous injection* |
| SC-69604 | No effect at tested dosages by intravenous injection |
| SC-71449 | No effect at tested dosages by intravenous injection |
| SC-72325 | Inhibited hyperalgesia 64% at 30 minutes |
| SC-73770 | Inhibited hyperalgesia 72% at 30 minutes |

*Higher dosage levels and other routes of administration were not tested for any of the compounds.

### Example 173

Analgesia provided by intravenous SC-72325 was evaluated over time in the carrageenan model.

### Example 174

Analgesia provided by intramuscular injection of SC-72325 was evaluated over time in the carrageenan model in comparison to the anti-inflammatory drug ketorolac.

### Example 175

To determine whether the SOD catalyst compounds provide analgesia by some action on the prostaglandin-leukotriene system, release of prostaglandin PGE2 was measured in rat paw exudate from the carrageenan model as well as in spinal cord fluid. Saline was used as a non-inflamed control and the anti-inflammatory ketorolac was used as a positive anti-inflammatory control. SC-72325 did not significantly reduce release of PGE2 compared to the carrageenan-injected but untreated rats. Ketorolac treated rats had levels of PGE2 release similar to non-carrageenan injected animals.

### Example 176

Mice were treated twice a day with either saline (naive) or morphine (s.c., 10 mg/kg) for a period of 4 days to induce tolerance. For comparison, a dose of 10 mg, or less than 0.15 mg/kg every 4 to 10 hours, is a morphine dosage routinely prescribed for the 70 kg. human adult with severe pain. On day 5, all mice received a subcutaneous challenge dose of 3 mg./kg morphine and the level of analgesia was measured 30 minutes later. Dose response measurements in normal mice have indicated that a challenge dose of 3 mg/kg would elicit 90% analgesia in naive or non-tolerant mice when assessed by the standard hot plate test. In this example, mice that were treated with morphine for 4 days showed a decreased analgesic effect from morphine on day 5 when compared with the naive mice. Tolerance to morphine was eliminated in mice that were treated with the superoxide dismutase catalyst SC-72325 administered intraperitoneally.

Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A method for treating, preventing, inhibiting, or reversing inflammatory disease in a subject, the method comprising administering a subject in need thereof a therapeutically effective amount of a non-proteinaceous catalyst for the dismutation of superoxide.

2. A method according to claim 1, wherein the non-proteinaceous catalyst for the dismutation of superoxide is a pentaaza-macrocyclic ligand complex represented by the following formula: 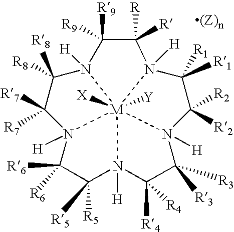
wherein R, R′, R₁, R′₁, R₂, R′₂, R₃, R′₃, R₄, R′₄, R′₅, R₆, R′₆, R₇, R′₇, R₈, R′₈, R₉ and R′₉ independently are selected from the group consisting of hydrogen and substituted or unsubstituted alkyl, alkenyl, alkynyl, cycloalkyl, cycloalkenyl, cycloalkylalkyl, cycloalkylcycloalkyl, alkylcycloalkyl, cycloalkenylalkyl, alkenylcycloalkyl, alkylcycloalkenyl, alkenylcycloalkenyl, heterocyclic, aryl and aralkyl radicals, or
R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉, together with the carbon atoms to which they are attached independently form a substituted or unsubstituted saturated, partially saturated or unsaturated cyclic ring structure having 3 to 20 carbon atoms; or
R or R′, R₁ or R′₁, and R₂ or R′₂, R₃ or R′₃ and R₄ or R′₄, R₅ or R′₅ and R₆ or R′₆, R₇ or R′₇, and R₈ or R′₈, and R₉ or R′₉, together with the carbon atoms to which they are attached independently form a substituted or unsubstituted nitrogen-containing heterocycle having 2 to 20 carbon atoms provided that when the nitrogen containing heterocycle is an aromatic heterocycle that does not have a hydrogen attached to the nitrogen, the hydrogen attached to the nitrogen in the macrocycle and the R groups attached to the same carbon atoms of the macrocycle are absent;
R and R′, R₁ and R′₁, R₂ and R′₂, R₃ and R′₃, R₄ and R′₄, R₅ and R′₅, R₆ and R′₆, R₇ and R′₇, R₈ and R′₈ and R₉ and R′₉, together with the carbon atom to which they are attached independently form a substituted or unsubstituted saturated, partially saturated or unsaturated ring structure having 3 to 20 carbon atoms; or
two of R, R′, R₁, R′₁, R₂, R′₂, R₃, R′₃, R₄, R′₄, R₅, R′₅, R₆, R′₆, R₇, R′₇, R₈, R′₈, R₉, and R′₉ attached to different carbon atoms of the macrocycle are bound to form a strap structure of the formula
—(CH₂)ₓ--M--(CH₂)_{w}--L--(CH₂)_{z}--J--(CH₂)_{y}—
wherein w, x, y and z independently are integers from 0 to 10 and M, L and J are independently selected from the group consisting of alkyl, alkenyl, alkynyl, cycloalkyl, aryl, heteroaryl, alkaryl, alkheteroaryl, aza, amido, ammonium, thio, sulfonyl, sulfinyl, sulfonamido, phosphonyl, phosphinyl, phosphino, phosphonium, keto, ester, carbamyl, ureido, thiocarbonyl, borate, borane, boraza, silyl, siloxy and silaza radicals, and combinations thereof; wherein X, Y and Z are pharmaceutically acceptable counterions or together are a pharmaceutically acceptable polydentate ligand, or are independently attached to one or more of the R groups and n is an integer from 0 to 3.

3. A method according to claim 2, wherein the pentaaza-macrocyclic ligand complex is represented by the following formula:

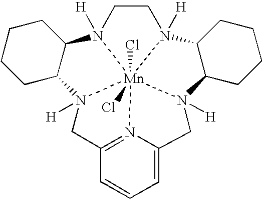

4. A method according to claim 1, wherein the subject is a mammal.

5. A method according to claim 4, wherein the mammal is a human.

6. A method for treating, preventing, inhibiting, or reversing arthritis in a subject, the method comprising administering a subject in need thereof a therapeutically effective amount of a non-proteinaceous catalyst for the dismutation of superoxide.

7. A method according to claim 6, wherein the non-proteinaceous catalyst for the dismutation of superoxide is a pentaaza-macrocyclic ligand complex represented by the following formula: 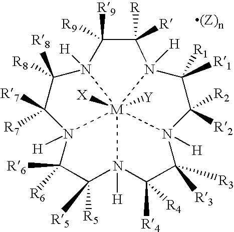
wherein R, R′, R₁, R′₁, R₂, R′₂, R₃, R′₃, R₄, R′₄, R′₅, R₆, R′₆, R₇, R′₇, R₈, R′₈, R₉ and R′₉ independently are selected from the group consisting of hydrogen and substituted or unsubstituted alkyl, alkenyl, alkynyl, cycloalkyl, cycloalkenyl, cycloalkylalkyl, cycloalkylcycloalkyl, alkylcycloalkyl, cycloalkenylalkyl, alkenylcycloalkyl, alkylcycloalkenyl, alkenylcycloalkenyl, heterocyclic, aryl and aralkyl radicals, or
R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉, together with the carbon atoms to which they are attached independently form a substituted or unsubstituted saturated, partially saturated or unsaturated cyclic ring structure having 3 to 20 carbon atoms; or
R or R′, R₁ or R′₁, and R₂ or R′₂, R₃ or R′₃ and R₄ or R′₄, R₅ or R′₅ and R₆ or R′₆, R₇ or R′₇, and R₈ or R′₈, and R₉ or R′₉, together with the carbon atoms to which they are attached independently form a substituted or unsubstituted nitrogen-containing heterocycle having 2 to 20 carbon atoms provided that when the nitrogen containing heterocycle is an aromatic heterocycle that does not have a hydrogen attached to the nitrogen, the hydrogen attached to the nitrogen in the macrocycle and the R groups attached to the same carbon atoms of the macrocycle are absent;
R and R′, R₁ and R′₁, R₂ and R′₂, R₃ and R′₃, R₄ and R′₄, R₅ and R′₅, R₆ and R′₆, R₇ and R′₇, R₈ and R′₈ and R₉ and R′₉, together with the carbon atom to which they are attached independently form a substituted or unsubstituted saturated, partially saturated or unsaturated ring structure having 3 to 20 carbon atoms; or
two of R, R′, R₁, R′₁, R₂, R′₂, R₃, R′₃, R₄, R′₄, R₅, R′₅, R₆, R′₆, R₇, R′₇, R₈, R′₈, R₉, and R′₉ attached to different carbon atoms of the macrocycle are bound to form a strap structure of the formula
—(CH₂)ₓ--M--(CH₂)_{w}--L--(CH₂)_{z}--J--(CH₂)_{y}—
wherein w, x, y and z independently are integers from 0 to 10 and M, L and J are independently selected from the group consisting of alkyl, alkenyl, alkynyl, cycloalkyl, aryl, heteroaryl, alkaryl, alkheteroaryl, aza, amido, ammonium, thio, sulfonyl, sulfinyl, sulfonamido, phosphonyl, phosphinyl, phosphino, phosphonium, keto, ester, carbamyl, ureido, thiocarbonyl, borate, borane, boraza, silyl, siloxy and silaza radicals, and combinations thereof; wherein X, Y and Z are pharmaceutically acceptable counterions or together are a pharmaceutically acceptable polydentate ligand, or are independently attached to one or more of the R groups and n is an integer from 0 to 3.

8. A method according to claim 7, wherein the pentaaza-macrocyclic ligand complex is represented by the following formula:

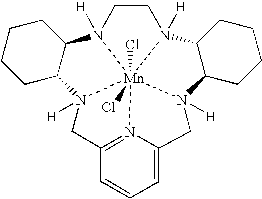

9. A method according to claim 6, wherein the arthritis is selected from the group consisting of rheumatoid arthritis and osteoarthritis.

10. A method according to claim 6, wherein the subject is a mammal.

11. A method according to claim 10, wherein the mammal is a human.
